SEQUENCE LISTING (10 sequences)

SEQ ID NO: 1 - protein, 352 aa, Aspergillus oryzae
MRVTTLSTALFALTSTAVSAPTAGSSSPGLEVKLTQIDNTRVKAVVKNTGSEEVSFVHLNFFKDAGPVKKVSVYRGQDEVQFEGIKRRLRSSGITKEAVTSLGAGETLEDEFDIASTSDLASGGPVSIRSHGFVPIVVDGKITGYIPYKSNDLTVNVDGGKAAKVTKALSQLTRRTEVTDCKGDAESSLTTALSNAAKLANQAAEAAESGDESKFEEYFKTTDQQTRTTVAERLRAVAKEAGSTSGGSTTYHCNDPYGYCEPNVLAYTLPSKNEIANCDIYYSELPPLAQKCHAQDQATTTLHEFTHAPGVYQPGTEDLGYGYDAATQLSAQDALNNADSYALYANAIELKC

SEQ ID NO: 2 - protein, 402 aa, Aspergillus oryzae
MQSIKRTLLLLGAILPAVLGAPVQETRRAAEKLPGKYIVTFKPGIDEAKIQEHTTWATNIHQRSLERRGATGGDLPVGIERNYKINKFAAYAGSFDDATIEEIRKNEDVAYVEEDQIYYLDGLTTQKSAPWGLGSISHKGQQSTDYIYDTSAGEGTYAYVVDSGVNVDHEEFEGRASKAYNAAGGQHVDSIGHGTHVSGTIAGKTYGIAKKASILSVKVFQGESSSTSVILDGFNWAANDIVSKKRTSKAAINMSLGGGYSKAFNDAVENAFEQGVLSVVAAGNENSDAGQTSPASAPDAITVAAIQKSNNRASFSNFGKVVDVFAPGQDILSAWIGSSSATNTISGTSMATPHIVGLSLYLAALENLDGPAAVTKRIKELATKDWKDVKGSPNLLAYNGNA

SEQ ID NO: 3 - protein, 375 aa, Aspergillus oryzae
HASADAWASSKAISSTSSPSSFKMRFISVSSLLLALAPALNAVPVEVAGSAQGLDVTLSQVGNTRIKAWKNTGSEDVTFVHLNFFKDAAPVQKVSLFRNATEVQFQGIKQRLITEGLSDDALTTLAPGATIEDEFDIASTSDLSEGGTITINSNGLVPITTDNKVTGYIPFTSNELSIDVDAAEAASVTQAVKILERRTKVTSCSGSRLSALQTALRNTVSLARAAATAAQSGSSSRFQEYFKTTSSSTRSTVAARLNAVANEAASTSSGSTTYYCSDVYGYCSSNVLAYTLPSYNIIANCDLYYSYLPALTSTCHAQDQATTTLHEFTHAPGVYSPGTDDLGYGYSAATALSASQALLNADTYALFANAVNLNC

SEQ ID NO: 4 - protein, 9 aa, Aspergillus oryzae
TEVTDCKGD

SEQ ID NO: 5 - protein, 9 aa, Aspergillus oryzae
GLTTQKSAP

SEQ ID NO: 6 - protein, 10 aa, Aspergillus oryzae
TKVTSNSGSR

SEQ ID NO: 7 - protein, 12 aa, Aspergillus oryzae
ARGVALARGARG

SEQ ID NO: 8 - protein, 9 aa, Aspergillus oryzae
HEEFEGRAS

SEQ ID NO: 9 - protein, 9 aa, Aspergillus oryzae
KRIKELATK

SEQ ID NO: 10 - protein, 4 aa, Artificial Sequence
  Chemically Synthesized
RVRR

(19) United States
(12) Patent Application Publication
(10) Pub. No.: US 2009/0068174 A1
(43) Pub. Date: Mar. 12, 2009

(54) THERAPEUTIC ALKALINE PROTEASE COMPOSITIONS AND USE IN FACILITATING THE TRANSPORT OF AGENTS ACROSS THE GASTROINTESTINAL MUCOSAL LINING

(75) Inventors: Michael J. Parmely, Kansas City, MO (US); Rohit Medhekar, Springfield, MO (US); Anthony Collier, Forsyth, MO (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Kansas University Medical Center Research Institute, Inc., Kansas City, KS (US)

(21) Appl. No.: 11/853,880

(22) Filed: Sep. 12, 2007

(51) Int. Cl. A61K 39/395; A61K 38/48; A61K 39/00; A61K 31/7088; A61K 31/7105; A61K 31/711; A61K 38/00; A61P 43/00

(52) U.S. Cl. 424/133.1; 424/94.63; 424/130.1; 424/184.1; 514/44; 514/2; 424/141.1; 424/139.1

(57) ABSTRACT

A method of treating an inflammatory condition involving TNF-α in a mammal by administering to a patient a composition with an effective amount of an isolated alkaline protease in an amount effective to inactive TNF-α. The invention also involves compositions, including pharmaceutical compositions containing an isolated alkaline protease in an amount effective to inactive TNF-α especially those from Aspergillus oryzae and/or serve as a transepithial carrier.

### FIELD OF THE INVENTION

The present invention relates generally to a methods of treating mammalian disease utilizing protelotyic enzymes of plant, animal, and/or microbial origin and their use in facilitating the transport of agents across the gastrointestinal mucosal lining. In particular, this invention relates to the therapeutic value of an alkaline protease isolated from the filamentous fungus Aspergillus oryzae for the treatment of inflammatory disorders mediated by TNF-α and its use in facilitating the transport of agents across the gastrointestinal mucosal lining.

### BACKGROUND OF THE INVENTION

### Therapeutic Alkaline Protease Compositions

Inflammatory bowel disease (IBD) is a collection of chronic disorders that include Crohn's disease, ulcerative colitis, and celiac disease. The incidence of these diseases has been increasing in developed countries over the past four decades. Recent advances in our understanding of the immunopathogenic mechanisms underlying these conditions have afforded new therapeutic approaches that target specific components of the inflammatory process. Cytokines, including tumor necrosis factor-α (TNF-α), are now known to play central roles in many forms of IBD as evidenced by the efficacy of anti-TNF-α drugs in their treatment. However, these therapies have a number of shortcomings, not the least of which is their costs.

Crohn's disease (CD) has an estimated incidence in North America approaching 200 cases/100,000 per year, a rate that has increased 8-10-fold since the 1960s. The prevalence of the disease is approximately 1 in 500 Americans. Despite drug therapy, up to 70% of CD patients undergo corrective surgery, and relapse after conventional therapies (corticosteroids, azathioprine, or methotrexate) is common. Recent advances in the use of biological therapies (e.g., anti-TNF-α monoclonal antibody; INFLIXIMAB™) have dramatically improved outcomes. However, the cost of treating a single CD patient with Infliximab ranges from $25,000 to $30,000 per year, excluding the cost of clinic visits necessary for intravenous injection of the drug. In addition, the systemic delivery of anti-TNF-α biologicals has been associated with an increased susceptibility to mycobacterial pneumonia, indicating the important role the cytokine plays in protective immune responses to intracellular microbial pathogens. Azathioprine and corticosteroids have similar side effects due to their nonspecific immunosuppressive activities. Adverse allergic reactions to INFLIXIMAB (infusion reactions) have also been reported.

CD has a clear immunological component initiated by innate immune responses to microbial flora. The chronic nature of CD is maintained by the persistent activation of inflammatory cells, including Th1 lymphocytes and submucosal macrophages. The cytokines interleukin-12 (IL-12), interferon-γ (IFN-γ), and tumor necrosis factor-α (TNF-α) play central roles in disease pathogenesis, and affected intestinal tissues of CD patients show marked elevations in the levels of TNF-α mRNA. Inducing the apoptosis of Th1 cells with the immunosuppressive agent azathioprine or neutralizing the activity of TNF-α with the monoclonal antibody INFLIXIMAB are both effective therapies for this disease in human beings. Similar cytokine-driven chronic inflammation characterizes ulcerative colitis, although the key mediators are not as clearly defined. In addition, proteases derived from microorganisms such as Aspergillus oryzae modify the course of inflammation and other body processes by selectively interacting with pro-inflammatory cytokines.

Proteolytic enzymes have a number of commercial applications and constitute one of the largest industrial classes of enzymatic proteins. Commercially important proteases are derived from plant, animal, and microbial sources and are available either as semi-purified or recombinant preparations. Proteases derived from Bacillus and Aspergillus species are among the most frequently used microbe-derived products and are often supplied as mixtures of several different enzymes. As such, these formulations can be active over a wide pH range and can show broad substrate specificities. Among the most common industrial and commercial applications of microbial proteases are their use in detergents, leather processing, and food production (e.g., cheese production, wheat gluten digestion, soy sauce production, debittering of food components, and aspartame production). For example, the alkaline protease of Aspergillus oryzae is an effective flavor-enhancing agent in the manufacture of soy sauce.

Proteolytic enzymes from animal or plant sources, such as trypsin, chymotrypsin, pepsin, papain, and bromelain, have utility as digestive enzymes.

Proteolytic enzymes also have been used in anti-inflammatory compositions. For example, proteolytic enzymes such as bromelain, papain, trypsin, and chymotrypsin have been traditionally used as anti-inflammatory agents, usually in the form of buccal tablets. In particular, microbial protease formulations such as Wobenzym N; Phlogenzym; Mulsal; and Wobe-Mugos E have been used as anti-inflammatories. Zhou et al. (1983) describes a method of intraduodenal injection of neutral peptidase isolated from Bacillus subtilis which showed a strong anti-inflammatory effect with low toxicity in a rat model.

Further, proteases from Aspergillus oryzae have been used in therapeutic methods as disclosed in U.S. Pat. No. 6,413,512 (Jul. 2, 2002) Houston et al., which describes crude preparations containing a mixture of Aspergillus oryzae proteases. Thus proteases, especially from Aspergillus oryzae, are not pathogenic in humans and are safe for the treatment of inflammatory conditions including gastrointestinal diseases. However, mixtures of proteases that are non-specific can affect patients broadly and a more specific therapy and more pure preparations of proteases are needed.

### The Transport of Agents Across the Gastrointestinal Mucosal Lining

Conventional means for delivering active agents to their intended targets (e.g., human organs, tumor sites) are often severely limited by the presence of biological, chemical, and physical barriers. Typically, these barriers are imposed by the environment through which delivery must take place, the environment of the target for delivery, or the target itself.

Biologically active agents are particularly vulnerable to such barriers. Oral delivery to the circulatory system for many biologically active agents would be the route of choice for administration to animals if not for physical barriers such as the mucosal gastrointestinal lining, lipid bi-layers, and various organ membranes that are relatively impermeable to certain biologically active agents, but which must be traversed before an agent delivered via the oral route can reach the circulatory system. Additionally, oral delivery is impeded by chemical barriers such as the varying pH in the gastrointestinal (GI) tract and the presence in the oral cavity and the GI tract of powerful digestive enzymes. U.S. Pat. No. 6,916,489 (Jul. 12, 2005) Milstein et al.

To date, many therapeutic compounds are discarded because no delivery systems are available to ensure that therapeutic titers of the compounds will reach the appropriate anatomical location or compartment(s) after administration and particularly oral administration. Furthermore, many existing therapeutic agents are underutilized for their approved indications because of constraints on their mode(s) of administration. Additionally, many therapeutic agents could be effective for additional clinical indications beyond those for which they are already employed if there existed a practical methodology to deliver them in appropriate quantities to the appropriate biological targets.

Broad spectrum use of prior delivery systems has been precluded, however, because: (1) the systems require toxic amounts of adjuvants or inhibitors; (2) suitable low molecular weight cargos (e.g., active agents) are not available; (3) the systems exhibit poor stability and inadequate shelf life; (4) the systems are difficult to manufacture; (5) the systems fail to protect the active agent (cargo); (6) the systems adversely alter the active agent; or (7) the systems fail to allow or promote absorption of the active agent. See U.S. Pat. No. 6,916,489 (Jul. 12, 2005) Milstein et al.

Oral bioavailability remains a crucial problem for the majority of pharmacologically active compounds under development. During the past decade it was observed that a vast amount of drugs share intestinal absorption pathways with those for nutrients. Currently, several of these transport routes (e.g. bile acid and peptide carrier systems) are under investigation for their potential utilization in drug delivery. As a result, our knowledge about substrate specificity and structure-transport relationships has significantly increased. Nevertheless, our understanding at the molecular level of these transport mechanisms is limited, making it difficult to predict recognition of a (new) drug by carrier-mediated mechanisms a priori.

The oral bioavailability of proteolytic enzymes (e.g. trypsin, chymotrypsin, papain and bromelain) has been studied since their introduction as anti-inflammatory, anti-edematose and immunostimulating agents. Oral enzyme absorption remains controversial because in the blood proteolytic enzymes bind to antiproteinases and therefore are difficult to quantify with immunological, enzymatic, and radiochemical methods. Therefore, the amounts of enzymes absorbed in vivo tend to be underestimated (immunological, enzymatic, methods) or overestimated (radiochemical methods). Quantification as an objective parameter, however, is pivotal for any bioavailability study. Regardless of these analytical difficulties, it is presently accepted that proteolytic enzymes, when orally administered, can be detected in the blood plasma, at least to some extent, in their intact, biologically active form. The proteolytic enzymes trypsin, chymotrypsin, papain and bromelain are believed to increase the permeability of the mucosal epithelium and, hence, facilitate their own absorption by a mechanism of self-enhanced paracellular diffusion. See Kolac et al. (1996) European Journal of Pharmaceutics and Biopharmaceutics 42(4): 222-232.

Many therapeutic and diagnostic agents are difficult to effectively administer because of their failure to cross the gastrointestinal lining. The absorption of drugs from the gastrointestinal tract is one of the important determinants for oral bioavailability. Further, as many patients may take several different kinds of drugs at the same time, it is possible that drug interactions at the intestinal absorption level are caused by the inhibition of transporters in the intestine. Therefore there is still a need in the art for simple, inexpensive delivery systems which are easily prepared and which can deliver a broad range of active agents to their intended targets, especially in the case of pharmaceutical agents that are to be administered via the oral route. Alkaline protease may be used to be administered to increase the bioavailability of agents, especially via oral administration.

### BRIEF SUMMARY OF THE INVENTION

It is an object of the present invention to provide a method of treating and/or prevention of inflammation which involves TNF-α in mammals. Another object of the present invention is to provide a method of treating and/or prevention of the recurrence of mammalian inflammatory diseases which involve TNF-α. Yet another object of the present invention is to provide a method of treating and/or prevention of the symptoms of mammalian inflammatory disease which involve TNF-α. Specifically, the object of the invention is the treatment of inflammatory disorders mediated by TNF-α. Another object of this invention includes a method of improving bioavailability of orally administered drugs. The invention further covers a method of screening for enhancers of bioavailability of orally administered drugs. The invention also covers application of the bioenhancer in pharmaceutical compositions for oral delivery of drugs, thereby providing novel and improved pharmaceutical compositions.

These and other objects of the invention are met by one or more of the following embodiments.

In one embodiment, this invention provides a method of treating anti-inflammatory condition involving TNF-α in a mammal comprising administering to said mammal a composition comprising an isolated alkaline protease in an amount effective to inactivate TNF-α. The inflammatory conditions suitable for treatment by administration of the composition comprising alkaline protease include but are not limited to diseases, conditions, maladies, illnesses that are related to, mediated by, caused by, exacerbated, aggravated, or the symptoms worsened by TNF-α. In particular, inflammatory conditions suitable for treatment by administration of the composition comprising alkaline protease include but are not limited to ulcerative colitis, asthma, Parkinson's disease (PD), cardiovascular disease, Crohn's disease (CD), multiple sclerosis (MS), irritable bowel syndrome (IBS), inflammatory bowel disease (IBD), irritable bowel disease, Alzheimer's disease (AD), infection, soft tissue injury, encephalitis, Graves' disease, myasthenia gravis, rheumatoid arthritis (RA), scleroderma, acute rheumatic fever, Kawasaki disease (KD), cachexia syndrome, acute pancreatitis, and chronic heart failure (CHF).

In another embodiment, the invention is directed to a use of a composition comprising an isolated alkaline protease for medical therapy. The medical therapy according to this invention is preferred to be for inflammatory conditions, disorders, and diseases, most preferred wherein said conditions, disorders, and diseases involved TNF-α. In particular, inflammatory conditions suitable for treatment by administration of the composition comprising alkaline protease include but are not limited to ulcerative colitis, inflammatory bowel disease (IBD), asthma, cardiovascular disease, Crohn's disease (CD), multiple sclerosis (MS), irritable bowel syndrome (IBS), Alzheimer's disease (AD), Parkinson's disease (PD), infection, soft tissue injury, encephalitis, Graves' disease, myasthenia gravis, rheumatoid arthritis (RA), scleroderma, acute rheumatic fever, Kawasaki disease (KD), cachexia syndrome, acute pancreatitis, and chronic heart failure (CHF).

Preferably, the composition comprising alkaline protease does not include a 26 kDa protease or deuterolysin. Also, the composition comprising alkaline protease can consist essentially of an isolated alkaline protease in an amount effective to inactive TNF-α. Typically the composition may have an optimum proteolytic activity at about pH=8.0. Preferably, the composition will have a maximum proteolytic activity in the range of from about pH 6.0 to 10.0.

Preferably, the isolated alkaline protease in the composition will be an Aspergillus oryzae alkaline protease, more preferably the isolated Aspergillus oryzae alkaline protease comprises SEQ ID NO: 2. The isolated Aspergillus oryzae (A. oryzae) alkaline protease of the composition can be recombinantly produced in microbial, plant, insect, and mammalian cells, and/or microbial, plant, insect, and mammalian hosts. The recombinantly produced alkaline protease can be made as a fusion protein, preferably with a cleavable linkage, and most preferably as a secreted fusion protein with a cleavable linkage.

In one embodiment, the composition comprising alkaline protease effective to treat inflammatory conditions is administered orally, injected, inhaled, or via suppository. Preferably, the composition is administered orally or buccally. Further said composition can comprise a pharmaceutically acceptable carrier, excipient, diluent, or solution. And, said composition can be a food supplement, a nutritional supplement, or a food product. The compositions suitable for use in the instant invention may comprise alkaline protease, consisting of alkaline protease, and/or consist essentially of alkaline protease (e.g., composition suitable for this invention may contain alkaline protease as the only active ingredient for anti-inflammatory treatment, wherein the balance of the composition is non-active ingredients—e.g., carriers, excipients, diluents, fillers).

In one embodiment, the composition comprising alkaline protease facilitates transport of therapeutic and diagnostic agents including but not limited to adjuvant, antibiotic, antibody, antigen, diagnostic agent, DNA vaccine, drug, gene-delivery vector, gene, macromolecule, nanoparticle, nucleic acid, ribonucleic acid, peptide, peptide vaccine, prodrug, protein, or vaccine. In this embodiment, the alkaline protease may act synergistically with the administered therapeutic or diagnostic agent to lower the amount required to be an effective dosage and/or minimize (or lessen) side effects of said administered agents.

In another embodiment, the invention encompasses a method of facilitating transport of an agent across a mucosal membrane in a patient comprising administering a composition comprising an alkaline protease and an agent to a patient, wherein the uptake of the agent is increased relative to uptake in the absence of said alkaline protease. In one embodiment, the aforementioned agent is an adjuvant, antibiotic, antibody, antigen, diagnostic agent, DNA vaccine, drug, gene-delivery vector, gene, macromolecule, nanoparticle, nucleic acid, ribonucleic acid, peptide, peptide vaccine, prodrug, protein, or vaccine. Additionally, the alkaline protease increases the bioavailability of the agent.

In another embodiment, the isolated alkaline protease is an Aspergillus oryzae alkaline protease, in particular, the isolated Aspergillus oryzae alkaline protease can comprise the amino acid sequence of SEQ ID NO: 2. The Aspergillus oryzae alkaline protease can be recombinantly produced by methods known in the art.

In another embodiment, the invention encompasses compositions comprising alkaline protease and an agent, including pharmaceutical compositions. The compositions described herein may comprise pharmaceutically acceptable excipients, diluents, carriers, solutions, and/or adjuvants. The compositions described herein may also be a food supplement, a nutritional supplement, and/or a food product. The compositions described herein may be administered orally. Further, the compositions described herein do not include a 26 kDa protease or deuterolysin.

A further embodiment is the use of a composition comprising an alkaline protease and an agent to facilitate transport of an agent across a mucosal membrane comprising administering wherein the uptake of the agent is increased relative to uptake in the absence of the alkaline protease. Another embodiment is the use of composition comprising an alkaline protease and an agent to facilitate transport of an agent across a mucosal membrane in the manufacture of a medicament to increase the uptake of the agent relative to its uptake in the absence of the alkaline protease.

In one embodiment, the isolated alkaline protease may be used to make antibody and/or antigen-binding fragment which specifically bind alkaline protease. The antibody can be a member of the immunoglobulin isotypes IgG, IgD, IgE, IgA, or IgM. In another embodiment, the antibody and antigen-binding fragment is chimeric, single-chain, or humanized. The antibody can also be monoclonal or polyclonal. The antigen-binding fragment can be F(ab)₂, Fab, Fv, and sFv. Further, the antibody or antigen-binding fragment may be specific for an Aspergillus oryzae alkaline protease, in particular, the alkaline protease amino acid sequence of SEQ ID NO: 2.

In another embodiment, the invention encompasses compositions comprising antibody and/or antigen-binding fragment which specifically bind alkaline protease, including pharmaceutical compositions. These compositions may comprise pharmaceutically acceptable excipients, diluents, carriers, solutions, and/or adjuvants.

The prior art provided mixtures of proteases that are non-specific which can affect patients broadly. Surprisingly, the present inventors have discovered a more specific therapy using more pure preparations of protease which retain the efficacy against TNF-α shown by prior art mixtures, but have less activity against other physiological components.

In particular, intact, enzymatically active A. oryzae proteases survive the mouse gastrointestinal tract, interact with the intestinal epithelium and translocate out of the intestinal lumen of the mouse to any significant extent. These alkaline proteases can be used for the treatment of local or extra-intestinal diseases in human beings as well as to facilitate the uptake of therapeutic and diagnostic agents across the mucosal gastrointestinal lining into the tissues and/or blood stream of a patient.

### BRIEF DESCRIPTION OF THE DRAWINGS

FIG. 1A-D

Schematic representation of four mechanisms of intestinal epithelium transport of small molecules and macromolecules. (A) Passive transcellular; (B) Passive paracellular; (C) Active carrier-mediated transcellular; (D) Transcytosis. In this figure, the rectangular boxes represent epithelial cells, and the top of the monolayer is the apical epithelial surface.

FIG. 2

A diagram of a production process to produce a protease powder containing alkaline protease.

FIG. 3

Source 30Q chromatography of a protease powder containing alkaline protease. The protease powder containing alkaline protease as prepared by Example 1 was loaded onto Source 30Q in 30 mM Tris, pH 8.0 and the unbound peak was collected. This peak had proteolytic activity (shaded area) and was designated Peak I. The remaining proteins were eluted with a linear 0-500 mM NaCl gradient in 30 mM Tris, pH 8.0. Peak IV also had protease activity when measured against protamine (shaded area).

FIG. 4

Source 30S Chromatography of Peak I from Source 30Q. 30Q Peak I was applied to Source 30S exchanger in 10 mM sodium acetate buffer, pH 5.5, and bound proteins were eluted with a linear NaCl gradient (0-250 mM) in the same buffer. Both eluted peaks had protease activity against the substrate protamine (shaded area) and were designated 30S Peak 1 and 30S Peak II.

FIG. 5

SDS-PAGE of purified A. oryzae proteases. Shown here are sample of protease powder containing alkaline protease as prepared by Example 1 (“Prep.”; lane 1) and three active protease peaks from ion exchange chromatography: 30Q Peak IV (lane 2), 30S Peak II (lane 3), and 30S Peak I (lane 4). The proteins were electrophoresed under reducing conditions on a 15% polyacrylamide SDS gel and visualized after staining with Coomassie Brilliant Blue. The major protein bands identified by squares had apparent molecular masses of 30 kDa (lane 2), 36 kDa (upper band of lane 3), 28 kDa (lower band of lane 3) and 26 kDa (lane 4). The gel was then blotted onto a PVDF membrane, and the indicated bands (dashed squares) were sequenced. Results of N-terminal amino acid sequencing are shown in Table 2. The letters below each lane refer to the ultimately determined identities of the proteins (Table 2).

FIG. 6

Amino acid sequence of Deuterolysin (SEQ ID NO: 1).

FIG. 7

Amino acid sequence of Alkaline Protease (SEQ ID NO: 2).

FIG. 8

Amino acid sequence of 26 kDa Protease (SEQ ID NO: 3).

FIG. 9

A scheme for the isolation of Aspergillus oryzae proteases.

FIG. 10 A-B

Elution profile for Source 30Q (Peaks I and IV) (A) and Elution profile from Source 30S (Peak I and II) (B).

FIG. 11 A-D

Effects of the purified A. oryzae proteases on human recombinant TNF-α: (A) protease powder prepared according to Example 1 (preparation); (B) alkaline protease; (C) 26 kDa protease; and (D) deuterolysin. 50 ng quantities of TNF-α were incubated at 37° C. for 18 hours with the indicated quantities of protease powder prepared according to Example 1, alkaline protease, 26 kDa protease or deuterolysin. Western blotting was then used to determine substrate specificity. C=control, buffer-treated TNF-α.

FIG. 12 A-D

Effects of purified A. oryzae proteases on the bioactivity of human TNF-α (A) protease powder prepared according to Example 1 (preparation); (B) alkaline protease; (C) 26 kDa protease; and (D) deuterolysin. 50 ng quantities of TNF-α were incubated at 37° C. for 24 hours with the indicated quantities (∘=buffer alone; =5 ng; ▪=10 ng) of: protease powder prepared according to Example 1 (preparation); alkaline protease; 26 kDa protease; and deuterolysin. The reactions were then stopped by addition of FBS and samples were added to C2C12 cells that had been costimulated with mouse IFN-γ. Twenty-four hours later, nitrite was measured in culture fluids.

FIG. 13 A-D

Effects of the purified A. oryzae proteases on human recombinant IFN-γ: (A) protease powder prepared according to Example 1 (preparation); (B) alkaline protease; (C) deuterolysin; and (D) 26 kDa protease. 50 ng quantities of human IFN-γ were incubated at 37° C. for 18 hours with the indicated quantities of preparation, alkaline protease, 26 kDa protease or deuterolysin. Western blotting was then used to determine substrate specificity. C=control, buffer-treated IFN-γ.

FIG. 14 A-B

Effects of the purified A. oryzae proteases on the Fc receptor-inducing activity of human IFN-γ. Samples of the cytokine were first treated with the indicated Pseudomonas or fungal proteases: Elastase (∘); Preparation (); Alkaline Protease (□); Deuterolysin (▪); and 26 kDa Protease (Δ). The treated samples were added to human promyelocytic U937 cells to induce expression of Fc receptors for IgG, which was detected by flow cytometry. The results are plotted either as a decrease in of mean channel fluorescence (A) or a decrease in % positive cells (B) plotted against increasing protease amounts.

FIG. 15 A-C

Degradation of mouse recombinant TNF-α: (A) or IFN-γ (B) following treatment with: the protease powder prepared according to Example 1 (preparation), purified alkaline protease (AP); Pseudomonas elastase (E); and buffered-treated control (C). The cytokines (100 ng each) were incubated at 37° C. overnight with the indicated quantities (ng) of enzymes, and then analyzed by Western blotting for evidence of degradation. A scan of AP-treated mouse TNF-α (left-hand Western blot) showing the decreasing density of the top band as a function of alkaline protease concentration (C).

FIG. 16 A-D

Effects of three purified A. oryzae proteases on the bioactivity of mouse TNF-α (A) protease powder prepared according to Example 1 (preparation); (B) alkaline protease; (C) 26 kDa protease; and (D) deuterolysin. 50 ng quantities of TNF-α were incubated at 37° C. for 24 hours with the indicated quantities of the protease powder prepared according to Example 1 or one of the three purified proteases. The reactions were then stopped by addition of FBS and samples were added to C2C12 cells that had been costimulated with mouse IFN-γ. Twenty-four hours later, nitrite was measured in culture fluids.

FIG. 17 A-B

The protease powder prepared according to Example 1 inactivates mouse recombinant TNF-α. 50 ng of the cytokine was treated for 24 hours with either 0 (□); 5 (∘); or 10 ng () of either Pseudomonas elastase (A) or protease powder prepared according to Example 1 (B). Then the residual bioactivity of the cytokine was measured by its ability to co-activate mouse C2C12 myoblast cells in the presence of excess IFN-γ for the production of NO (nitrite). Treatment of the mouse TNF-α with either protease led to a loss of its biological activity.

FIG. 18 A-D

Effects of three purified A. oryzae proteases on the bioactivity of mouse IFN-γ: (A) protease powder prepared according to Example 1 (preparation); (B) alkaline protease; (C) 26 kDa protease; and (D) deuterolysin. 50 ng quantities of mouse recombinant IFN-γ were incubated for 24 hours at 37° C. with either buffer or 2.5-10 ng of the indicated protease: (∘) Buffer; (▴) 2.5 ng; () 5 ng; (▪) 10 ng. IFN-γ bioactivity was determined as conduction of NO production by C2C12 cells.

FIG. 19

Titration of a rabbit antibody to A. oryzae alkaline protease against the immunizing peptide (A) or the authentic enzyme (B). Pre/Pres indicates the pre-immunization serum. PB indicates post-immunization bleeds 1-3, which were approximately 4 weeks apart. Binding to the two antigens was determined by ELISA.

FIG. 20

pH Profile of Protease Powder Prepared According to Example 1.

FIG. 21 A-C

TNF-α was first treated either with PBS, AP or D for 2 h, the reaction mixtures were inactivated by the addition of FBS, and the cytokine-protease mixtures were injected i.p. into mice. (A) One hour later the mice were euthanized, samples of their intestines were collected and fixed, and tissue sections were analyzed by immunohistology for the expression of activated caspase-3. These are the results are shown in which data are expressed in two forms: (B) the frequency distributions of sections expressing different levels of activated caspase-3; and (C) the percentages of tissue sections in which 10% or more of the villi stained for activated caspase-3. Treating TNF-α with increasing concentrations of AP, but not D, destroyed the ability of the cytokine to activate caspase-3 in intestinal epithelial cells.

FIG. 22 A-B

In this figure, various amounts of deutrolysin and alkaline protease were administered to mice 15 min prior to challenge with TNF-α. Intestinal tissues were again recovered 1 h later and the expression of activated caspase-3 was measured. (A) Pretreating mice with AP at doses of 1-10 μg blocked the action of TNF-α in vivo, whereas pretreatment with D was without a significant inhibitory effect. (B) The ability of AP to provide protection against the apoptotic effects of TNF-α in vivo was limited in its duration. Thus, pretreatment of the mice for up to 30 min prior to TNF-α challenge prevented caspase-3 activation, whereas delaying cytokine challenge beyond 30 min diminished the protective effect of the protease.

### DETAILED DESCRIPTION OF THE INVENTION

### Definitions

Unless otherwise indicated, all terms used herein have the same meaning as they would to one skilled in the art.

The term “alkaline protease”, as used in this specification and claims, indicates a protease or protease mixture, which has maximum proteolytic activity in the range of from about pH 6-10, most preferably of about pH ˜8. The alkaline protease has maximum proteolytic activity wherein it is shows at least 80% of its maximal activity level from about pH 6-10. Preferably, the alkaline protease has a maximum activity at about pH 7-9. Also, the alkaline protease only shows one major activity peak between pH 6-10 and no peaks at pH 4-6.

It must be noted that, as used in this specification and the appended claims, the singular forms “a,” “an,” and “the” include plural referents unless the context clearly dictates otherwise. Thus, for example, reference to “an antioxidant” includes a mixture of one or more antioxidants, reference to “a vitamin” includes reference to one or more of such vitamins, and reference to “a microbial protease” includes references to one or more of such microbial proteases.

The Aspergillus species according to the invention are preferably Aspergillus aculeatus, Aspergillus auricomus, Aspergillus caesillus, Aspergillus conicus, Aspergillus ficuum, Aspergillus flavus, Aspergillus foetidus, Aspergillus fumigatus, Aspergillus japonicus, Aspergillus nidulans, Aspergillus niger, Aspergillus nomius, Aspergillus ochraceus, Aspergillus oryzae, Aspergillus ostianus, Aspergillus parasiticus, Aspergillus parasiticus, Aspergillus phoenicus, Aspergillus phoenicus, Aspergillus restrictus, Aspergillus sake, Aspergillus soja (Aspergillus sojae), Aspergillus sydowii, Aspergillus tamari, Aspergillus terreus, Aspergillus ustus, and/or Aspergillus versicolor. The most preferred species for use in the instant invention is Aspergillus oryzae (A. oryzae).

“Increased” or “increase” as used herein, refers broadly to a quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater. In particular, the term “increase”, as used herein, refers broadly to make greater, as in number, size, strength, or quality; add to; and/or augment. “Increase”, as used herein, also encompasses expand, extend, prolong, augment, enlarge, grow, develop, and/or swell. “Increase”, as used herein, additionally encompasses where a given parameter (e.g., level, amount, size, scope, duration, weight) is greater, as in number, size, strength, or quality, than it once was. Furthermore, the “increase” in any number, size, strength, or quality of a given parameter may be determined as between to two or more time points, especially if before or after a treatment, event, or administration of an agent or composition. Further, “increase” refers broadly to significant or detectable, functionally, analytically, and/or clinically, changes in the number, size, strength, or quality of a given parameter in question. In particular, increase refers to a change in levels and/or activity of a cytokine (e.g., TNF-α) or alkaline protease (e.g., AP).

“Therapy” or “therapeutic” as used herein, refers broadly to treating a disease, arresting or reducing the development of the disease or its clinical symptoms, and/or relieving the disease, causing regression of the disease or its clinical symptoms. Therapy encompasses prophylaxis, prevention, treatment, cure, regimen, remedy, minimization, reduction, alleviation, and/or providing relief from a disease, signs, and/or symptoms of a disease. Therapy encompasses an alleviation of signs and/or symptoms in patients with ongoing disease signs and/or symptoms, e.g. of inflammation. Therapy also encompasses “prophylaxis” and “prevention”. Prophylaxis includes preventing disease occurring subsequent to treatment of a disease in a patient or reducing the incidence or severity of the disease in a patient. The term “reduced”, for purpose of therapy, refers broadly to the clinical significant reduction in signs and/or symptoms. Therapy includes treating relapses or recurrent signs and/or symptoms, e.g. of inflammation. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease.

Therapy can be for patients with risk factors, at risk patients in a susceptible population, patients with a history of disease, patients with symptoms, patients with signs, patients with signs but no symptoms, and patients with symptoms but no signs. Therapy can also be for patients without risk factors, not at risk, patients not in a susceptible population, patients no history of disease, patients with no symptoms, patients without signs. Therapy can alleviate, allay, abate, assuage, curtail, decrease, ease, lessen, lighten, make better, make healthy, mitigate, mollify, pacify, relieve, rehabilitate, remedy, repair, and/or soothe a disease, disease signs, and/or disease symptoms.

“Treating” or “treatment”, as used herein, refers broadly to a course of therapy where signs and/or symptoms are present in the patient. The term “reduced”, for purpose of therapy, refers broadly to clinically significant reduction in signs and/or symptoms. Treatment includes treating chronic disease (“maintenance”) and acute disease. Treatment can be for patients with risk factors, at risk patients in a susceptible population, patients with a history of disease, and/or patients with symptoms, patients with signs. Treatment can alleviate, allay, abate, assuage, curtail, decrease, ease, lessen, lighten, make better, make healthy, mitigate, mollify, pacify, relieve, rehabilitate, remedy, repair, and/or soothe a disease, disease signs, and/or disease symptoms.

“Prophylaxis”, as used herein, refers broadly to a course of therapy where signs and/or symptoms are not present in the patient, are in remission, or were previously present in a patient. The term “reduced”, for purpose of therapy, refers broadly to the clinical significant reduction in the incidence of signs and/or symptoms. Prophylaxis includes preventing disease occurring subsequent to treatment of a disease in a patient or reducing the incidence or severity of the disease in a patient. Prophylaxis encompasses “prevention” and as used herein, is a subset under “therapy” and refers broadly to inhibiting the disease, arresting the development of the disease or its clinical symptoms, and/or relieving the disease, causing regression of the disease or its clinical symptoms. Prevention also preferably includes prophylaxis as preventing or reducing incidence or severity of disease in a patient. Further, prevention includes treating patients who can potentially develop the disease, especially patients who are susceptible to the disease, e.g. members of a patent population, those with risk factors, or at risk for developing the disease. Prevention also includes preventing relapses or the recurrence of signs and/or symptoms, e.g. inflammation.

“Signs” of disease, as used herein, refers broadly to any abnormality indicative of disease, discoverable on examination of the patient; an objective indication of disease, in contrast to a symptom, which is a subjective indication of disease.

“Symptoms” of disease as used herein, refers broadly to any morbid phenomenon or departure from the normal in structure, function, or sensation, experienced by the patient and indicative of disease.

“Therapeutically effective amount” as used herein, refers broadly to the amount of a compound that, when administered to a patient for treating a disease, is sufficient to effect such treatment for the disease. The therapeutically effective amount can be an amount effective for prophylaxis, and/or an amount effective for prevention. The therapeutically effective amount can be an amount effective to reduce inflammation, an amount effective to prevent inflammation, to reduce the severity of inflammation, to eliminate inflammation, to slow the development of inflammation, to prevent the development of inflammation, and/or effect prophylaxis of inflammation. The “therapeutically effective amount” can vary depending on the disease and its severity and the age, weight, medical history, predisposition to conditions, preexisting conditions, etc., of the patient to be treated. The term “effective amount” is taken to be synonymous with “therapeutically effective amount” for purposes of this invention.

“Prophylatically effective amount” as used herein, refers broadly to the amount of a compound that, when administered to a patient for prophylaxis or prevention of a disease or reoccurrence of a disease, is sufficient to effect such prophylaxis or prevention for the disease or reoccurrence. The prophylatically effective amount is, in particular, an amount effective for prophylaxis, and/or an amount effective for prevention. The prophylatically effective amount can be an amount effective to prevent inflammation, to reduce the severity of inflammation if it reoccurs, to slow the development of inflammation, to prevent the development of inflammation, and/or effect prophylaxis of inflammation. The “prophylatically effective amount” can vary depending on the disease and its severity and the age, weight, medical history, predisposition to conditions, preexisting conditions, etc., of the patient to be treated. The term “effective amount” is taken to be synonymous with “prophylatically effective amount” for purposes of this invention.

“Administration” as used herein, refers broadly to any means by which a composition is given to a patient.

“Patient” as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient can be a clinical patient such as a human or a veterinary patient such as a companion, domesticated, livestock, exotic, or zoo animal. Animals can be mammals, reptiles, birds, amphibians, or invertebrates.

“Mammal” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, chimpanzees, chinchillas, cattle, dogs, goats, gorillas, horses, humans, lemurs, llamas, mice, non-human primates, pigs, rats, sheep, shrews, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C., which is hereby incorporated by reference.

“Antibody,” as used herein, refers broadly to immunoglobulin molecules and immunologically active portions of immunoglobulin molecules (e.g., molecules that contain an antigen binding site that specifically binds an antigen). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody. Preferably, antibodies that specifically bind to alkaline protease do not cross-react with other antigens. Antibodies that specifically bind to alkaline protease can be identified, for example, by immunoassays or other techniques known to those of skill in the art.

“Transport” as used herein, refers broadly to the translocation of agents from one physiological compartment to another, usually across a cell-cell barrier such as the muscoal gastroinestinal lining. “Transport” includes but is not limited to carry, convey, cross, move, passage, transfer, transference, and/or transit. For instance, transport includes when an agent travels, transits or crosses a compartment, usually a membrane, cell lining, facia, sinus, to carry, move, and/or convey from one place to another, in particular, from one physiological compartment to another (e.g., from the stomach into the bloodstream across the gastrointestinal mucosal barrier). This includes but is not limited to crossing the gastrointestinal lining, the blood-brain barrier, the uterine blood sinus, the respiratory mucosal lining, and/or the urinary tract lining,

“Facilitate”, as used herein, refers broadly to the increase and/or improvement of transport from one physiological compartment to another, usually across a cell-cell barrier. For instance, “facilitate” includes aid, increase, assist, expedite, promote, improve, allow, permit, and/or enhance transport of an agent by means of administration of alkaline-protease.

“Bioavailability”, as used herein, refers broadly to the availability of a drug to an animal following administration and can be used interchangeably with “systemic exposure” (e.g. the bioavailability of a drug is expressed as the systemic exposure of a cell to drugs).

M-cells (microfold cells) are antigen sampling cells that are found in the epithelium of the gut-associated lymphoid tissue, or Peyer's Patch.

### Effects of Protease Mixtures

Proteases can modify the course of inflammation and other normal or disease processes via selective inactivation by means of proteolysis of protein and peptide mediators of disease or normal body functions. Proteases cleave biologically relevant proteins and peptides destroying their biological activities in a selective fashion that reflects the primary, secondary, tertiary, and quaternary structure of these proteins and peptides and the specificity of the proteases.

The specificity of the proteases can be demonstrated by comparing two or more proteases with regard to their ability to inactivate a given biologically active protein. The specificity of the proteases can be demonstrated by comparing the effect of a given protease on two or more biologically active proteins (substrates).

In general, proteases can be separated from one another and, when separated, show unique properties, including distinct specificities for biologically active proteins and peptides. For instance, individual proteases purified from Aspergillus oryzae (A. oryzae) show unique specificity for biologically active proteins. This property of a protease's specificity affords the opportunity to limit adverse effects by administering a purified reagent with a limited range of activities in vivo. The corollary is that a mixture limited to one or a few proteases may have fewer or no serious side effects.

This property affords the opportunity to combine purified proteases in various ratios to create compositions with identifiable specificities under selected application conditions. This property affords the opportunity to enhance the activity of the proteases as it is well-recognized that proteases can act synergistically on protein and peptide substrates. This property endows mixtures of proteases with wider ranges of specificity and stability in vivo, if that is a desirable characteristic for a particular application.

### Therapeutic Utilities of Alkaline Protease

In inflammatory conditions, chemokines attract inflammatory cells to the lungs and promote pulmonary damage. Orally administered proteases are effective at limiting the action of these peptide chemokines. The therapeutic utility of the alkaline protease is based on observations in vitro and in a mouse model that Aspergillus oryzae (A. oryzae) alkaline protease selectively cleaves and inactivates a known mediator of inflammation, tumor necrosis factor-α (TNF-α). Alkaline protease administered as either a mixture or a purified protease cleaves and inactivates TNF-α. The alkaline protease described herein may be used in a method of treating an inflammatory condition mediated by TNF-α in a mammal comprising administering to said mammal a composition comprising an effective amount of an isolated alkaline protease in an amount effective to inactive TNF-α by means of cleavage.

For purposes of this invention, mouse models of inflammatory bowel disease (IBD) are suitable experimental vehicles for testing the effectiveness of alkaline protease on mucosal inflammatory diseases. Using an animal model of acute TNF-α-induced intestinal inflammation, the inventors have shown that alkaline protease has significant therapeutic use as an anti-TNF-α reagent. Aspergillus oryzae (A. oryzae) alkaline protease provides substantial value in both the prevention of TNF-α-dependent inflammatory lesions in the gut and the treatment of ongoing bowel inflammation in mouse models of IBD. Aspergillus oryzae alkaline protease has protective effects on the induction of IBD in the hapten-induced mouse model when the alkaline protease is administered systemically or by the oral route. Aspergillus oryzae alkaline protease can lessen intestinal lesions when administered to mice with established IBD. Aspergillus oryzae alkaline protease can be used to treat IBD in mammals that spontaneously develop the disease.

The bioactivity of mouse TNF-α can be decreased in vivo by prior treatment of mice with Aspergillus oryzae proteases. Aspergillus oryzae alkaline protease diminishes the enterocyte damage associated with the systemic administration of recombinant mouse TNF-α. A mouse TNF-α-induced gastroenteritis model using markers of enterocyte apoptosis as a measure of the proinflammatory activity of the cytokine validates the utility of Aspergillus oryzae alkaline protease.

In addition, purified Aspergillus oryzae alkaline protease, when administered systemically to mice, can protect against TNF-α-induced apoptosis of epithelial cells lining the terminal villi of the duodenum. The lesions induced in this animal model appeared first within cells that constitute the anatomical barrier between the host and the intestinal lumen. This is relevant for the development of IBD, as altered epithelial barrier function is a critical early step in disease pathogenesis.

The mouse model system can be used to establish the therapeutic value of A. oryzae alkaline protease in the treatment of acute and chronic inflammatory diseases and provide a pathophysiological framework within which the gastrointestinal transport of the proteases following oral delivery can be studied. Further, Aspergillus oryzae alkaline protease is held to be an effective agent for the prevention and treatment of Crohn's disease and other TNF-α-mediated inflammatory diseases of the bowel, particularly via oral administration. The inflammatory conditions suitable for treatment by the methods taught herein include but are not limited to acute pancreatitis, acute rheumatic fever, allergic asthma, Alzheimer's disease (AD), asthma, autoimmune diseases, cachexia syndrome, cardiovascular disease, chronic heart failure (CHF), Crohn's disease (CD), encephalitis, Graves' disease, infection, inflammatory bowel disease (IBD), irritable bowel syndrome, Kawasaki disease (KD), multiple sclerosis (MS), myasthenia gravis, Parkinson's disease (PD), rheumatoid arthritis (RA), scleroderma, soft tissue injury, and ulcerative colitis.

In another embodiment, an affliction, ailment, condition, disease, disorder, illness, infirmity, inflammation, injury, malady, misery, pathosis, sickness, and/or syndrome mediated by TNF-α can be treated by administration of alkaline protease compositions as described herein. Further, an affliction, ailment, condition, disease, disorder, illness, infirmity, inflammation, injury, malady, misery, pathosis, sickness, and/or syndrome dependent upon TNF-α can be treated by administration of alkaline protease compositions as described herein. Further, an affliction, ailment, condition, disease, disorder, illness, infirmity, inflammation, injury, malady, misery, pathosis, sickness, and/or syndrome involving TNF-α can be treated by administration of alkaline protease compositions as described herein.

The preferred A. oryzae alkaline protease composition has the following properties: (1) resistance to inactivation by exposure to pH=2, pepsin, and trypsin; (2) heat stable at 37° C. for 3 hours; (3) ability to cleave and inactivate TNF-α; (4) stable in dry form at room temperature (˜25° C.) for 2 years; and (5) a pH optimum of from about pH ˜6-10, most preferably from about pH ˜7-10, most preferably at about pH ˜8. The preferred alkaline protease acts therapeutically by means of decreasing or preventing TNF-α mediated inflammation.

### Alkaline Protease Activation of Beneficial Proteins

The alkaline protease described herein can be used individually or in combination to treat inflammatory processes that are known to involve distinct protein mediators. The action of alkaline proteases is not limited to inactivation, but can also be applied to the activation of beneficial proteins. Further, adverse effects can be limited through the use of a purified reagent with a limited range of activities in vivo. Additionally, purified proteases can be combined in various ratios to create known compositions with identifiable specificities for applications under selected conditions. The alkaline protease described herein specifically acts by means of cleaving and inactivating TNF-α, which may lead to the activation of beneficial proteins.

### Animal Models of Inflammatory Bowel Disease (IBD)

There currently exist nearly two dozen experimental rodent models of IBD, which differ in terms of the major cellular and molecular mediators of tissue injury and the nature and distribution of the lesions. All converge on common pathogenic pathways in which either Th1 (IFN-γ, IL-12 and TNF-α) or Th2 (IL-4 and IL-5) cytokines predominate. Recent studies indicate that the Th1 models (e.g., hapten-induced colitis) accurately reflect the pathology of CD, while Th2 models are appropriate for studying ulcerative colitis. The importance of TNF-α as a central mediator of Th1-type IBD is illustrated both by the ability of agents that block TNF-α action to decrease pathology and the occurrence of spontaneous colitis in animals genetically engineered to over-express either TNF-α or the TNF receptor.

The colitis induced by the hapten trinitrobenzene sulfonic acid (TNBS) provides an art-accepted model with the following qualities: (i) TNF-α is an essential mediator of disease progression as evidenced by antibody neutralization studies and the failure to establish the disease in mice lacking the TNF-α receptor; (ii) AP treatment can be administered at controlled time points relative to the induction of the disease in this model; (iii) the disease is immunologically mediated and the pathway of induction has been carefully defined; (iii) the initial pathological changes include a loss of villus architecture and damage to intestinal epithelial cells overlying lymphoid aggregates that resembles the aphthoid lesions found in human CD. Mucosal ulceration follows and progresses to transmural intestinal inflammation mimics the inflammatory lesions seen in advanced CD.

The present findings surprisingly indicate that purified A. oryzae AP, when administered systemically to mice, can protect against TNF-α-induced apoptosis of epithelial cells lining the terminal villi of the duodenum. This finding supported earlier observations that AP hydrolyzed and inactivated mouse TNF-α in vitro. More importantly, the results constitute evidence that an A. oryzae protease can interfere with the induction of an inflammatory process in an art-accepted animal model in which the initiator of tissue damage (e.g., the cytokine TNF-α) is known. The lesions induced in this animal model appeared first within cells that constitute the anatomical barrier between the host and the intestinal lumen. This is relevant for the development of IBD, as altered epithelial barrier function is now thought to be a critical early step in disease pathogenesis. Importantly, recent results indicate that mouse models of IBD are useful experimental vehicles for testing the effectiveness of AP and other proteases on mucosal inflammatory diseases in general.

Additional mouse models of IBD (especially CD) include the spontaneously arising diseases in C3H/HeJBir and SAMP1/Yit strains of mice. Both models have the advantage of showing a natural disease progression that does not require chemical induction. The SAMP1/Yit mouse shows lesion distribution primarily in the ileum; in the C3H/HeJBir mouse, the lesions are primarily found in the colon and cecum. In both models, blocking the effects of TNF-α substantially reduces pathology.

### Protease Effects on Gastrointestinal Function

Growth factors, cell surface receptors, extracellular matrix proteins, cell anchoring structures that maintain tissue organization, molecules that mediate cell migration within the tissues and cell-cell communication, nutrients, and host defense mediators are all candidate protein and peptide mediators of gastrointestinal physiology and disease, and may be acted upon by exogenous proteases provided in the diet or under a therapeutic regimen. The alkaline protease described herein can act by means of cleaving and inactivating TNF-α, which may lead to the desired effects on gastrointestinal function.

Alternatively, proteases can activate or inactivate biologically important substrates in the gut lumen, at the surface of the mucosal epithelium or within gastrointestinal-associated tissues. The alkaline protease can be used in purified form or in pre-determined compositions (mixtures), each of which have unique properties against specific substrates. Compositions of alkaline proteases each have specific applications based on their unique synergistic compositions and stability properties in vivo.

Alkaline proteases pass beyond the mucosal surface in an enzymatically-active form and gain access to the epithelium, subepithelium or intestinal-associated tissues where they act on protein or peptide mediators of normal cell and tissue function as well as mediators of disease. Protein and peptide mediators of gastrointestinal physiology and disease that can be acted upon by exogenous alkaline proteases provided in the diet or under a therapeutic regimen include, but are not limited to, growth factors, cell surface receptors, extracellular matrix proteins, cell anchoring structures that maintain tissue organization, molecules that mediate cell migration within the tissues and cell-cell communication, nutrients, and host defense mediators. Thus, alkaline proteases can activate or inactivate biologically important substrates in the gut lumen, at the surface of the mucosal epithelium or within GI-associated tissues.

### Transepithelial Transport of Alkaline Protease

The epithelial cells lining the lumenal side of the gastrointestinal tract (GIT) are a major barrier to drug delivery following oral administration. The ability of a conventional drug, peptide, protein, macromolecule, nanoparticle system, or microparticle system to interact with one of these transport pathways may result in increased delivery of that drug or particle from the GIT to the underlying circulation.

Aspergillus oryzae alkaline protease, when administered by the oral route, can interact with the intestinal epithelium in a fashion that facilitates its transport out of the lumen of the gastrointestinal tract in an enzymatically active form. Intact, enzymatically active Aspergillus oryzae alkaline protease can survive the mouse gastrointestinal tract, interact with the intestinal epithelium, and translocate out of the intestinal lumen of the mouse to a significant extent.

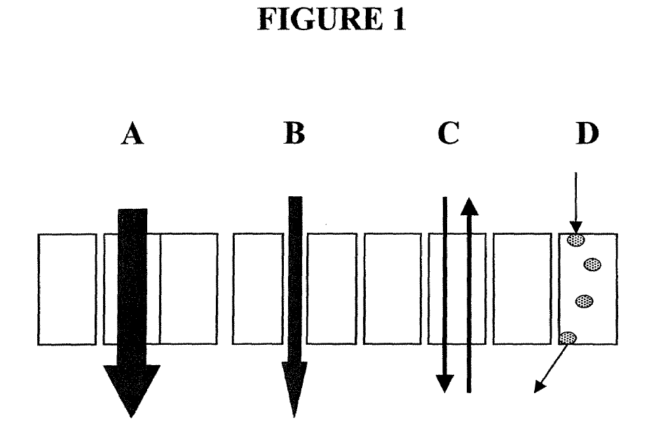

Without being bound to a particular theory, four mechanisms have been described by which alkaline protease can be transported across epithelial barriers, including that of the intestine: (A) passive transcellular; (B) passive paracellular; (C) active carrier-mediated transcellular; and (D) transcytosis (FIG. 1). FIG. 1A represents passive diffusion through the cell, which is typical of lipophillic drugs. By contrast, hydrophillic molecules typically travel through water-filled pores of the paracellular pathway (FIG. 1B), although tight junctions can restrict this route. Agents, such as surfactants and fatty acids, increase the movement of molecules across the epithelium by the paracellular route. Molecules that mimic nutrients can be transported by an active carrier-mediated mechanism (FIG. 1 C), and this general mechanism can transport in both directions. FIG. 1D depicts the most common route for transport of macromolecules excluded by other routes in an undisturbed epithelium and can involve receptor-mediated uptake (e.g., vitamin B₁₂). Although not limited to M cells (microfold cells), the nonvillus intestinal epithelium overlying the Peyer's patches is particularly efficient at using this mechanism to transport macromolecules, including dietary antigens. Without being committed to a single theory, it is believed that doses of alkaline protease described herein results in their uptake through the M cell epithelium and deposition within the Peyer's patches and mesenteric lymph nodes.

The inventors have discovered herein that the oral delivery of certain proteolytic enzymes can have positive health benefits, including the supplementation of the digestive enzyme function. Orally-administered proteases were usually believed not to survive the hostile conditions of the upper gastrointestinal tract, and the art does not teach a mechanism by which these macromolecules might exit the lumen of the alimentary canal. The present inventors found that orally administered proteases derived from the filamentous fungus Aspergillus oryzae survive within the gastrointestinal tract and associated tissues of the mouse. These proteases can cleave and inactivate mouse and human cytokines known to play important roles in inflammatory diseases. Thus, the inactivation of these cytokines provides a biologically relevant assay of the effects of the fungal proteases on known inflammatory mediators. Certain A. oryaze proteases, when administered by the oral route, interact with the intestinal epithelium in a fashion that facilitates their transport out of the lumen of the gastrointestinal tract in an enzymatically active form. The inventors have identified and characterized the biochemical and immunological properties of these proteases.

In another embodiment, the invention pertains to detection systems capable of monitoring the organ, tissue, and cellular distribution of proteases in the mouse and assessing the specific activity of any protease found to interact with cells and tissues of the gastrointestinal tract. Also, the present invention provides for the evaluation of the ability of purified A. oryzae proteases to translocate across intact monolayers of polarized mouse intestinal epithelial cells in vitro. The invention herein also may be used to define the distribution of protease within mouse tissues following administration by several routes, including the oral route, and characterize the biochemical, immunological, and catalytic properties of A. oryzae proteases distributed in extra-gastrointestinal tissues, including the blood circulation.

Alkaline protease can pass beyond the mucosal surface in an enzymatically-active form and gain access to the epithelium, subepithelium, or intestinal-associated tissues where it acts on TNF-α mediators of normal cell and tissue function, as well as mediators of disease. Therefore administering certain treatments of disease by the oral, rather than parenteral, route may be preferred.

### Alkaline Protease as a Transepithelial Carrier

In addition, the alkaline protease can have effects on the normal permeability of the gastrointestinal tract epithelium which can extend to other compounds, including other therapeutic and diagnostic agents (“piggy-back” or adjuvant effects). These therapeutic agents may be covalently attached to the alkaline protease or non-covalently associated with alkaline protease. Such therapeutic and diagnostic agents may be co-administered in separate compositions or combined into a single composition. Thus, the alkaline protease may facilitate the uptake and transport of other orally administered therapeutic and diagnostic agents. In general, the uptake of the agent is greater with the alkaline protease than without it.

A preferred method of the invention for administering a carrier entity to a mammal having intestinal epithelium comprises contacting the intestinal epithelium with an alkaline protease of the invention in the presence of the carrier entity, such that the carrier entity is transported into or across the intestinal epithelium or into or across a preferred region of the intestine such as M-cells or Peyer's patches.

The entity and the alkaline protease can be administered together (e.g., as part of an entity-ligand complex) or discretely or separately. Oral administration is most preferred, but other modes of administration requiring transepithelial transport to reach the target tissue are also acceptable (e.g., rectal administration).

Of course, the ability of the alkaline protease of the invention to target certain cells of the intestinal epithelium also makes the ligands suitable for targeting pharmaceutical agents to the cells themselves for therapy or prophylaxis. Alternatively, the alkaline protease may be delivered to selected tissue sites (e.g., the immune environment of the Peyer's patches) by taking advantage of dedicated protein transport mechanisms (e.g., across M cells).

Applications of these concepts to disease include pharmacological and immunological considerations in addition to the treatment of inflammatory, autoimmune, or neoplastic diseases, such as increasing the permeability of the gastrointestinal epithelium to other agents, including other peptides or proteins or any compound used in therapy. Using this increased permeability, many drugs currently administered by the parenteral route (e.g., insulin) become available in an oral form. This specialized form of protein transport provides for the use of proteases as modulators of mucosal immune responses within the gut or other submucosal tissues (e.g., the lung). An example might be the use of proteases as immune enhancers. Mucosal vaccine development is currently limited by the lack of mucosal adjuvants (agents that increase the immunogenicity of a vaccine protein) that do not have significant adverse effects on the host. Thus, protease therapy is an effective adjunct to oral drug treatment in general or at least in the case of specific classes of drugs.

Further, translocating proteases beyond the epithelial surface increase the efficacy of vaccines delivered by the oral route. Without being committed to a single theory, mechanisms include modifying natural immune modulators, promoting antigen processing and presentation or altering lymphocyte signaling for growth and differentiation through growth factors, such as cytokines and chemokines. Thus, alkaline protease acts as an immunoenhancing agent to promote oral immunization (vaccination) against a host of mucosal vaccine products. Translocating proteases beyond the epithelial surface alters a number of disease states by affecting cell surface receptors for protein or peptide mediators of those diseases. Examples include autoimmune conditions in the GI or GI-associated tissues that require inflammatory cell activation through a cell surface receptor (including GI cancers).

In an embodiment of the invention, a composition comprising alkaline protease is administered wherein the alkaline protease acts as a carrier to facilitate the uptake of other components of the composition, especially when administered orally, buccally, in a gum, or intraoral patch or pouch. Thus, the alkaline protease compositions described herein can facilitate the uptake and transport of other agents including but not limited to adjuvants, antibiotics, antibodies, antigens, diagnostic agents, DNA vaccines, drugs, gene-delivery vectors, genes, macromolecules, nanoparticles, nucleic acids and ribonucleic acids, peptides, peptide vaccines, prodrugs, proteins, and vaccines.

As contemplated by this invention, this invention, adjuvants include but are not limited to 3 De-O-acylated monophosphoryl lipid A (MPL™), aluminum hydroxide, aluminum phosphate, aluminum salt adjuvants, calcium phosphate, CpG, Freund's Complete Adjuvant, Freund's Incomplete Adjuvant, Gerbu Adjuvant, Hunter's Titermax, Montanide ISA Adjuvant, Nitrocellulose adsorbed protein, QS-21 (STIMULON™), and Ribi's Adjuvant.

In another embodiment of this invention, antibiotics include but are not limited to penicillin, amoxicillin, cephalosporin, griseofulvin, erythromycin, amphotericin B, polymyxin B, bacitracin, rifamycin, neomycin, streptomycin, tetracycline, vancomycin, gentamicin, kanamycin, chlortetracycline, oxytetracycline, doxycycline, erythromycin, azithromycin, clarithromycin, clindamycin, ciprofloxacin, levofloxacin, moxifloxacin, doxycycline, linezolid, and combinations thereof.

As contemplated by this invention, antibodies include but are not limited to monoclonal, polyclonal, single-chain, humanized, chimeric antibodies, and/or antibody fragments thereof. Also, antibodies include therapeutic antibodies such as anti-TNF-α monoclonal antibody (e.g., INFLIXIMAB™).

As contemplated by this invention, diagnostic agents include but are not limited to antibodies, nucleic acids, imaging agents, radioisotopes, dyes, contrast agents, ultrasound-enhancing agents, optical-enhancing agents, fluorescent compounds or molecules and enhancing agents (e.g. paramagnetic ions) for magnetic resonance imaging (MRI) as well as molecules that are needed to make such agents detectable.

As contemplated by this invention, drugs include but are not limited to analgesics, anti-migraine agents, anti-coagulant agents, anti-emetic agents, cardiovascular agents, anti-hypertensive agents, narcotic antagonists, chelating agents, anti-anginal agents, chemotherapy agents, sedatives, anti-neoplastics, prostaglandins and antidiuretic agents. Representative drugs also include but are not limited to analgesics (e.g. fentanyl, sufentanil, butorphanol, buprenorphine, levorphanol, morphine, hydromorphone, hydrocodone, oxymorphone, methadone, lidocaine, bupivacaine, diclofenac, naproxen and paverin); anti-migraine agents (e.g. sumatriptan and ergot alkaloids); anti-coagulant agents (e.g. heparin and hirudin); anti-emetic agents (e.g. scopolamine, ondansetron, domperidone and metoclopramide); cardiovascular agents, anti-hypertensive agents and vasodilators (e.g. diltizem, clonidine, nifedipine, verapamil, isosorbide-5-mononitrate, organic nitrates and agents used in treatment of heart disorders); sedatives (e.g. benzodiazepines and phenothiozines); narcotic antagonists (e.g. naltrexone and naloxone); chelating agents (e.g. deferoxamine); anti-diuretic agents (e.g. desmopressin and vasopressin); anti-anginal agents (e.g. nitroglycerine); anti-neoplastics (e.g. 5-fluorouracil and bleomycin); prostaglandins; and chemotherapy agents (e.g. vincristine).

As contemplated by this invention, gene-delivery vectors include but are not limited to recombinant viruses, gutless vectors, DNA molecules, viral vectors (e.g. adenovirus, adeno-associated virus, retroviruses, herpes simplex virus, and sindbus virus), and cationic lipid-coated DNA and DNA-dendrimers.

As contemplated by this invention, nanoparticles include but are not limited to nanospheres, nanorods, nanocups, and nanotubes.

As contemplated by this invention, nucleic acids and ribonucleic acids include but are not limited to antisense oligonucleotides, gene-correcting hybrid oligonucleotides, ribozymes, RNA interference (RNAᵢ) oligonucleotides, silencing RNA (siRNA) oligonucleotides, aptameric oligonucleotides, and triple-helix forming oligonucleotides.

As contemplated by this invention, peptides whose uptake may be facilitated by alkaline protease including, in particular, peptide hormones, which are not limited to insulin, calcitonin, calcitonin gene regulating protein, atrial natriuretic protein, colony stimulating factor, betaseron, erythropoietin (EPO), interferons (e.g. α, β, or γ interferon), somatropin, somatotropin, somatostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VITT, and interleukins (e.g. interleukin-2).

A vaccine preparation will contain at least one antigen, examples of antigens include but are not limited to tumor antigens, pathogen antigens and allergen antigens. “Pathogen antigens” are those characteristic of pathogens, such as antigens derived from viruses, bacteria, parasites or fungi.

Examples of important pathogens include vibrio choleras, enterotoxigenic E. coli, rotavirus, Clostridium difficile, Shigella species, Salmonella typhi, parainfluenza virus, influenza virus, Streptococcus mutans, Plasmodium falciparun, Staphylococcus aureus, rabies virus and Epstein-Barr virus.

Viruses contemplated by this invention include but are not limited to the following families: picronaviridae; caliciviridae, togaviridae; flaviviridae; coronaviridae; rhabodviridae; filoviridae; paramyxoviridae; orthomyxoviridae; bunyaviridae; arenaviridae; reoviridae; retroviridae; hepadnaviridae; parvoviridae; papovaviridae; adenoviridae; herpesviridae; and poxyviridae. These viruses, especially attenuated versions or otherwise modified versions that are not pathogenic, can also be modified to express targeting ligands on their surface and thus allow for enhanced vaccination.

Bacteria contemplated by this invention include but are not limited to: P. aeruginosa; E. coli; Klebsiella species; Serratia species; Pseudomanas species; P. cepacia; Acinetobacter species; S. epidermis; E. faecalis; S. pneumonias; S. aureus; Haemophilus species; Neisseria species; N. meningitidis; Bacterodies species; Citrobacter species; Branhamella species; Salmonelia species; Shigella species; S. Lesteria species, Pasteurella multocida; Streptobacillus species; S. pyogenes; Proteus species; Clostridium species; Erysipelothrix species; Spirillum species; Fusospirocheta species; Treponema pallidum; Borrelia species; Actinomycetes; Mycoplasma species; Chlamydia species; Rickettsia species, Spirchaeta; Legionella species; Mycobacteria species; Urealplasma species; Streptomyces species; Trichomoras species; and P. mirabilis.

Parasites include but are not limited to: Plasmodium falciparum, P. vivax, P. ovale, P. malaria; Toxoplasma gondii; Leishmania mexicana, L. tropica, L. major, L. aethiopica, L. donovani, Trypanosoma cruzi, T. brucei, Schistosoma mansoni, S. haematobium, S. japoniurn; Trichinella spiralis; Wuchereria bancrofti; Brugia malayli; Entamoeba histolytica; Enterobus vermiculoarus; Taenia solium, T. saginata, Trichomonas vaginitis, T. hominis, T. tenax; Giardia lamblia; CrypLosporidium parvum; Pneumocytis carinii, Babesia bovis, B. divergens, B. microti, Isospore belli, L. hominis; Dientamoeba fragiles; Onchocerca volvulus; Ascaris lumbricoides; Necator americanis; Ancylostorna duodenale; Strongyloides stercoralis; Capillaria philippinensis; Angiostrongylys cantonensis; Hymenolepis nan; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phtirius pubis; and Dermatobia hominis.

For purposes of this invention, fungi in general include but are not limited to: Crytpococcus neoformans; Blastomyces dematitidis; Aiellomyces dermatitidis Histoplasfrai capsulatum; Coccidiodes immitis; Candids species, including C. albicans, C. tropicalis, C. parapsilosis, C. guilliermondii and C. krusei, Aspergillus species, including A. fumigatus, A. flavus and A. niger, Rhizopus species; Rhizomucor species; Cunnighammella species; Apophysomyces species, including A. saksenaea, A. mucor and A. absidia; Sporothrix schenckii, Paracoccidioides brasiliensis; Pseudallescheria boydii, Torulopsis glabrata; and Dermatophyres species.

Further, antigens that are allergens can be haptens, or antigens derived from pollens, dust, molds, spores, dander, insects and foods. Specific examples include the urusiols of Toxicodendron species and the sesquiterpenoid lactones.

Industrial applications of alkaline protease as a transepithelial carrier include pharmacological and immunological uses in addition to use in the manufacture of medicaments for the treatment of inflammatory, autoimmune, or neoplastic diseases.

Translocating the proteases beyond the epithelial surface is advantageous in the use of treating inflammatory conditions affecting the intestinal epithelium or submucosa mediated by protein or peptide inflammatory mediators (e.g., inflammatory bowel disease, Crohn's disease). Translocating proteases beyond the epithelial surface can increase the efficacy of vaccines delivered by the oral route. Thus, proteases can act as immunoenhancing agents to promote oral immunization (vaccination) against a host of mucosal vaccine products.

Translocating proteases beyond the epithelial surface can alter a number of disease states by affecting cell surface receptors for protein or peptide mediators of those diseases. Examples might include autoimmune conditions in the GI or GI-associated tissues that require inflammatory cell activation through a cell surface receptor. This includes GI cancers (liver, pancreatic, bowel).

In general, the alkaline protease and the agent which it facilitates the transport of can be co-administered, sequentially administered, separately administered. Alternately, the alkaline protease can be administering within seconds, minutes, hours, or days before or after administration of the agent that the person is seeking to facilitate the transport from one physiological compartment to another.

Proteases of Aspergillus oryzae

Four of the proteases produced by Aspergillus oryzae are listed in Table 1. They represent a range of catalytic types, including serine and metalloproteases. All belong to the endopeptidase category and in the aggregate act over a broad pH range. However, they differ in their pH optima, thermostability properties, and substrate specificities. The acid protease of A. oryzae (Table 1) exists in two similar, but disparate molecular forms, which have been shown to differ from one another in that the larger form is heavily glycosylated.

TABLE 1
Properties of Selected Proteases from Aspergillus oryzae

| | Deuterolysin | | | |
|---|---|---|---|---|
| | (Neutral | Alkaline | Serine | Aspergillopepsin O |
| Property | Protease II) | Protease | Carboxypeptidase | (Acid Protease) |
| Catalytic Type | Zn | Serine | Serine protease | Aspartic protease |
| | metalloprotease | protease | | |
| Reported | Hemoglobin | Hemoglobin, | | Hemoglobin |
| Substrate | (low), | Milk casein, | | Milk casein |
| Specificities^{a} | Milk casein | Salmon | | Bovine pancreatic |
| | (low), | protamine | | pepsinogen |
| | Salmon | sulfate, | | |
| | protamine | CBZ-val-leu- | | |
| | sulfate (high), | lys | | |
| | Boc-arg-val-arg- | | | |
| | arg | | | |
| pH optimum | 5.0-7.0 | 7.0-10.0 | 4.5 | 3.0-5.0 |
| Approx. MW | 19 kDa | 28 kDa | 67 kDa | 32 kDa (63 kDa |
| | | | | glycosylated form) |
| Amino acids | 177 | 282 | | |
| pI | 4.53 | 6.53 | | 3.9 and 3.5 |
| Heat stability | Stable at 100° C., | Stable at | | Unstable above 55° C. |
| | Unstable at | 37° C. for 3 h | | |
| | 75° C. | | | |
| EC Number | 3.4.34.39 | 3.4.21.63 | | 3.4.23.6 |

^{a}Relative activities against these natural and synthetic substrates vary widely.

### Alkaline Proteases Sources

The isolated alkaline protease can be purified from cells that naturally express it, purified from cells that have been altered to express it (recombinant), or synthesized using standard methods known in the art. The alkaline protease can also be obtained from commercial sources as a purified or isolated enzyme, in a cultural supernatant of Aspergillus oryzae, or as part of a food grade protease blend available from and Amano Enzyme Inc. (Elgin, Ill.). Purified strains of Aspergillus oryzae are also commercially available from American Type Culture Collection (ATCC) (Manassas, Va.). Alkaline protease from Bacillus spp. is available from Sigma Aldrich (St. Louis, Mo.) The strains may be grown by using standard methods in the art and the alkaline protease can be purified using methods described herein and as known in the art.

Purification of Alkaline Proteases from Natural Sources

The isolated protease can be purified from cells that naturally express it using standard methods known in the art. A suitable protocol for purification is described in Examples 1 and 2 below, although the skilled person could readily develop alternative protocols based on standard biochemical principles. A protease according this invention should be “isolated” or “purified”. The proteases of the present invention can be purified to homogeneity or other intermediate degrees of purity. The level of purification will be based on the intended use. The critical feature is that the preparation allows for the desired function of the protease, even if in the presence of considerable amounts of other components. The preferred embodiment of an “isolated” or “purified” alkaline protease is where the alkaline protease component constitutes at least about 51% of the hydrolytic activity of the composition, measured against salmon protamine (nmoles Leucine/30 min./μg protein), and has a pH optimum equal to or greater than about pH˜8.0. Another embodiment of an “isolated” or “purified” alkaline protease is where it is the predominant protease in a composition, comprises at least 51% of the hydrolytic activity of the composition, wherein the specific activity of the “isolated” or “purified” alkaline protease has specific activity of at least about 900 for salmon protamine (nmoles Leucine/30 min./μg protein) and/or 100 for Val-leu-lys-pNA (mOD/min/μg protein). The specific activity may be determined by methods as taught herein and known in the art.

### Preparation of Alkaline Proteases by Chemical Synthesis

The isolated protease can be synthesized using known protein synthesis methods such as solid-phase synthesis of polypeptides or chemical ligation. Other methods for chemical synthesis known in the art are described in Coligan et al. (Eds.) (2003) Current Protocols in Protein Science Wiley, which is incorporated by reference.

The language, “substantially free of chemical precursors or other chemicals”, includes preparations of the protease in which it is at least 95% free of chemical precursors or other chemicals that are involved in its synthesis.

Preparation of Alkaline Proteases from Recombinant Sources

The isolated protease can be purified from cells that have been altered to express it (recombinant). DNA sequences encoding the protease may be inserted into an expression vector and then transformed (or transfected) in an appropriate host cell and/or expressed in a transgenic animal.

### Nucleic Acids

In particular, a nucleic acid molecule encoding the protease is cloned into an expression vector, the expression vector introduced into a host cell and the protein expressed in the host cell. The protease can then be isolated from the cells by an appropriate purification scheme using standard protein purification techniques which are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods suitable for this invention which are known in the art are described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

The present invention further provides isolated nucleic acid molecules that encode a protease of the present invention (cDNA, transcript, and genomic sequence). Such nucleic acid molecules will consist of, consist essentially of, or comprise a nucleotide sequence that encodes one of the alkaline protease of the present invention, an allelic variant thereof, or an ortholog or paralog thereof.

Moreover, an “isolated” nucleic acid molecule, such as a transcript/cDNA molecule, is free of other nucleic acids from the biological species in which the nucleic acid occurs in nature. Preferably, an “isolated nucleic acid” is substantially free of other cellular material, or culture medium when produced by recombinant techniques, or chemical precursors or other chemicals when chemically synthesized. However, the nucleic acid molecule can be fused to other coding or regulatory sequences and still be considered isolated.

Accordingly, the present invention provides protease that comprises of the amino acid sequences provided in SEQ ID NO: 2. The alkaline protease may also comprise of the nucleic acid sequence which encodes SEQ ID NO: 2. A protein comprises of an amino acid sequence when the amino acid sequence is the final amino acid sequence of the protein. Accordingly, the present invention provides protease that consists of the amino acid sequences provided in SEQ ID NO: 2. The invention may also consist of the nucleic acid sequence which encodes SEQ ID NO: 2. A protein consists of an amino acid sequence when the amino acid sequence is the final amino acid sequence of the protein.

In another embodiment, sequence variants of at least 70% sequence homology with SEQ ID NO: 2 that retain the biological activity and substrate specificity of the alkaline protease are suitable for this invention. In another embodiment, sequence variants of at least 80%, at least 85%, at least 90%, at least 95%, at least 98%, and/or at least 99% sequence homology with SEQ ID NO: 2 that retain the biological activity and substrate specificity of the alkaline protease are suitable for this invention. The comparison of sequences and determination of percent identity and similarity between two sequences can be accomplished using a mathematical algorithm as described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part 1, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991, all of the foregoing are incorporated by reference.

In another embodiment, sequence variants with over 70% sequence homology with SEQ ID NO: 2 that retain the biological activity and substrate specificity of the alkaline protease are suitable for this invention. In another embodiment, sequence variants with over 80%, with over 85%, with over 90%, with over 95%, with over 98%, and/or with over 99% sequence homology with SEQ ID NO: 2 that retain the biological activity and substrate specificity of the alkaline protease are suitable for this invention. The comparison of sequences and determination of percent identity and similarity between two sequences can be accomplished using a mathematical algorithm as described in Computational Molecular Biology, Lesk, A. M., ed., Oxford University Press, New York, 1988; Biocomputing: Informatics and Genome Projects, Smith, D. W., Ed., Academic Press, New York, 1993; Computer Analysis of sequence Data, Part 1, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991, all of the foregoing are incorporated by reference.

Further the genetic code is known to be redundant and several nucleic acids may code for SEQ ID NO: 2. Also, several nucleic acids may be deduced from the amino acid of SEQ ID NO: 2, preferably using the codon preference of the host organism. Any of these sequence variants of the alkaline protease as described herein and suitable for this invention retain the biological activity and substrate specificity of the alkaline protease as described herein.

### Vectors

The invention also provides vectors containing the nucleic acid molecules described herein. The term “vector” refers to a vehicle, preferably a nucleic acid molecule, which can transport the nucleic acid molecules. When the vector is a nucleic acid molecule, the nucleic acid molecules are covalently linked to the vector nucleic acid. The invention provides vectors for maintenance (cloning vectors) or vectors for expression (expression vectors) of the nucleic acid molecules. The vectors can function in prokaryotic or eukaryotic cells or in both (shuttle vectors). With this aspect of the invention, the vector includes a plasmid, single or double stranded phage, a single or double stranded RNA or DNA viral vector, or artificial chromosome, such as a BAC, PAC, YAC, or MAC. Plasmids suitable for this invention include but are not limited to pUC19, pBR322, pCMV, pSK Bluescript, pcDNA3, pcDNA3.1, pGEM, pGEX, pGST, pEGFP, and vectors disclosed in the Promega Complete Vector List, which is herein incorporated by reference.

The nucleic acid molecules can be inserted into the vector nucleic acid by methodology known in the art. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art. Appropriate cloning and expression vectors for prokaryotic and eukaryotic hosts, as well as protocols, are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods suitable for this embodiment are known in the art described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

### Host Cells

The vector containing the appropriate nucleic acid molecule can be introduced into an appropriate host cell for propagation or expression using well-known techniques. Host cells may be prokaryotic, including but not limited to bacterial cells, or eukaryotic, including but not limited to insect, fungal, mold, yeast, animal, and/or plant cells. Several host cells suitable for this invention are described New England BioLabs, Inc. (2004) 2005-2006 Catalog and Technical Reference, which is incorporated by reference.

The recombinant host cells are prepared by introducing the vector constructs described herein into the cells by techniques readily available to the person of ordinary skill in the art. These include, but are not limited to calcium phosphate transfection, cationic lipid-mediated transfection, conjugation, DEAE-dextran-mediated transfection, electroporation, infection, lipofection, protoplast transformation, transduction, and other techniques such as those found in Sambrook et al. (2001) Molecular Cloning: A Laboratory Manual (3^{rd} Ed.) Cold Spring Harbor Laboratory Press, which is incorporated by reference, and Ausubel et al. (2002) Short Protocols in Molecular Biology (5^{th} Ed.) Wiley, which is incorporated by reference.

While the mature proteins can be produced in bacteria, yeast, insect, plant, mammalian cells, and other cells under the control of the appropriate regulatory sequences, cell-free transcription and translation systems can also be used to produce these proteins using RNA derived from the DNA constructs described herein.

Where the peptide is not secreted into the medium, which is typically the case with proteases, the protein can be isolated from the host cell by standard disruption procedures, including freeze thaw, sonication, mechanical disruption, and the use of lysing agents. The peptide can then be recovered and purified by purification methods known in the art including but not limited to acid extraction, affinity chromatography, ammonium sulfate precipitation, anion exchange chromatography, cationic exchange chromatography, high performance liquid chromatography (HLPC), hydrophobic-interaction chromatography, hydroxylapatite chromatography, lectin chromatography, and phosphocellulose chromatography.

### Alkaline Protease Polypeptides

The present invention provides for an alkaline protease. In particular, the invention also provides for an alkaline protease, preferably from Aspergillus oryzae. The alkaline protease described herein has the following properties, it is about 29 kDa in size as determined by SDS-PAGE gel (if glycosylated, the alkaline protease can be as large as about 36 kDa, and may be any size between 28-36 kDa), has a pI of about 6.5, has a pH optimum from about 7-10, and has substrate specificity of protamine>casein and a specific activity of at least 900 salmon protamine (nmoles Leucine/30 min./μg protein) and at least 100 Val-leu-lys-pNA (mOD/min/μg protein measured at 405 nm).

The alkaline protease as described herein predominately cleaves TNF-α, and poorly cleaves IFN-γ (as shown in FIGS. 14-16).

The present invention further provides proteases that consist essentially of the amino acid sequences provided in SEQ ID NO: 2 and are encoded by nucleic acids which encode for SEQ ID NO: 2. A protein consists essentially of an amino acid sequence when such an amino acid sequence is present with only a few additional amino acid residues, for example from about 1 to about 100 or so additional residues, typically from 1 to about 20 additional residues in the final protein. The present invention further provides proteins that comprise of the amino acid sequences provided in SEQ ID NO: 2, and are encoded by nucleic acids which encode for SEQ ID NO: 2. The present invention further provides proteins that consist of the amino acid sequences provided in SEQ ID NO: 2, and are encoded by nucleic acids which encode for SEQ ID NO: 2.

Allelic variants, paralogs, fragments, orthologs, and non-naturally occurring variants of the protease of the present invention can readily be generated using recombinant techniques. Such variants include, but are not limited to deletions, additions and substitutions in the amino acid sequence of the protease peptide. For example, one class of substitutions are conserved amino acid substitutions. Such substitutions are those that substitute a given amino acid in a protease peptide by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent is found in Bowie et al. (1990) Science 247: 1306-1310, which is incorporated by reference. Any and all of the allelic variants, paralogs, fragments, orthologs, and non-naturally occurring variants of the protease as described herein retain the biological activity and substrate specificity of the alkaline protease.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. All of these methods are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods known in the art are described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

Further in accordance with this invention, the protease may contain amino acids other than the 20 amino acids commonly referred to as the 20 naturally occurring amino acids and still retain the biological activity and substrate specificity of the protease. Further, many amino acids, including the terminal amino acids, may be modified by natural processes, such as processing and other post-translational modifications, or by chemical modification techniques well known in the art. Known modifications include, but are not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, lipid attachment, methylation, myristoylation, oxidation, proteolytic processing, phosphorylation, prenylation, racemization, selenoylation, sulfation, transfer-RNA mediated addition of amino acids to proteins such as arginylation, and ubiquitination. The modifications as described herein and methods for adding them to the alkaline protease are known in the art as taught by Coligan et al. (Eds.) (2003) Current Protocols in Protein Science Wiley, which is incorporated by reference.

### Methods of Isolation of Alkaline Protease

The alkaline protease in this invention may be extracted, isolated, and/or purified from the host cells cultivated in a nutrient medium suitable for production of the protease using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the protease to be expressed and/or isolated. The cultivation takes place in a suitable nutrient medium comprising carbon and nitrogen sources and inorganic salts, using procedures known in the art. Suitable media are available from commercial suppliers or may be prepared according to compositions known in the art.

If the protease is secreted into the nutrient medium, the enzyme can be recovered directly from the medium. If the protease is not secreted, it is recovered from cell lysates. The cells expressing the alkaline protease is cultivated in a nutrient medium suitable for production of a polypeptide of interest using methods known in the art, many of which are described in Coligan et al. (Eds.) (2003) Current Protocols in Protein Science Wiley, which is incorporated herein by reference.

The protease may be recovered by methods known in the art including, but not limited to centrifugation, chromatography, evaporation, extraction, filtration, precipitation, preparative isoelectric focusing, and spray-drying. The recovered protease may then be further purified by a variety of chromatographic procedures including but not limited to affinity, chromatofocusing, gel filtration, hydrophobic, ion exchange, and size exclusion ion exchange. The recovered protease may then be further purified by a variety of electrophoretic procedures including but not limited to preparative isoelectric focusing (IEF). The recovered protease may then be further purified by a variety of differential solubility techniques including but not limited to ammonium sulfate precipitation. Other methods known in the art are described by Janson and Ryden (Eds) (1989) Protein Purification VCH Publishers, which is incorporated by reference. One suitable method for isolation of the alkaline protease is described in Examples 1-3.

The protease may be detected using methods known in the art that are specific for the protease. These detection methods may include use of specific antibodies, such as those taught in Example 10, the formation of an protease product, the disappearance of an alkaline protease substrate, or SDS-PAGE. For example, an alkaline protease assay may be used to determine the activity of the alkaline protease. Procedures for determining protease activity are known in the art.

### Compositions

The proteases described herein may be included in compositions. These compositions may be pharmaceutical, nutritional supplements, food additive, foodstuff, aqueous solutions, and gels.

“Composition” as used herein, refers broadly to any composition containing a therapeutic agent and/or agents. The composition can comprise a dry formulation, an aqueous solution, a paste formulation, an organic solution formulation, a colloid formulation, a cream formulation, a gel formulation, a jelly formulation, or a sterile composition. Compositions comprising the alkaline proteases described herein can be stored in freeze-dried form and can be associated with a stabilizing agent such as a carbohydrate including but not limited to fructose, sucrose, galactose, lactose, and maltose.

The composition of the present preferred embodiment is orally administered in capsule (hard or soft, coated or uncoated), tablet (coated or uncoated), powder or granule (coated or uncoated) or liquid (solution or suspension) form and dissolves easily in the stomach.

The composition of the present invention may also include additional ingredients such as other enzymes, analgesics, vitamins, minerals, antioxidants, bioflavonoids, proanthocyanidins, herbs, herbal extracts, and plant and animal concentrates.

A present preferred embodiment may also contain an effective amount of a non-prescription or prescription analgesic, including but not limited to acetaminophen, acetylsalicylic acid (aspirin), ibuprofen, indomethacin, ketoprofen, naproxyn, salicylates, and sulindac. Dosages of such analgesics should not exceed federal regulations.

A present preferred embodiment may also contain an effective amount of vitamins, including but not limited to vitamin A, vitamin B1, vitamin B2, vitamin B6, vitamin B12, vitamin C, vitamin D, vitamin E, vitamin K, niacin, pantothenic acid, folic acid, and biotin.

A present preferred embodiment may also contain an effective amount of minerals, including but not limited to calcium, chloride, chromium, copper, fluoride, iodine, iron, magnesium, manganese, molybdenum, phosphorus, potassium, selenium, sodium, and zinc.

A present preferred embodiment may also contain an effective amount of antioxidants, bioflavonoids, and proanthocyanidins. The preferred antioxidant is vitamin C (ascorbic acid) is provided in the composition in the form of calcium ascorbate, which is a buffered form of ascorbic acid. Other potent antioxidants are in the form of bioflavonoids and proanthocyanidins, including bioflavonoids such as quercetin (3.3′,4′,5,7-pentahydroxyflavone) and rutin (3-rhamnoglucoside of 5,7,3′,4′-tetrahydroxyflavonol).

In aspects of the invention the starting material may contain added material which will improve the properties of the resulting dry protease containing powder or products resulting here from. Useful additives include materials selected from acid agents, alkaline agents, anti-foaming agents, binders to other enzymes, biocides, carbohydrates, cellulose or derivatives thereof, coloring pigments, dispersants, inorganic minerals or clays, flavorings, preservatives, protease inhibitors, protease stabilizers, salts, sweeteners, viscosity regulating agents, and combinations thereof.

Calcium is provided in the present preferred embodiment as calcium citrate and calcium ascorbate because the mineral is optimally absorbed by the body from these two forms. Clinical experience with high doses of orally-administered microbial protease has resulted in muscle cramping in some patients. Oral administration of calcium in conjunction with microbial protease can be used to alleviate cramping.

### Pharmaceutical Compositions

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other means of administration.

A “pharmaceutical excipient” or a “pharmaceutically acceptable excipient” is a carrier, usually a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline protease can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The preferred forms of administration in the present invention are oral forms know in the art of pharmaceutics. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

### Nutritional Compositions

The compositions of the alkaline protease described herein may be used in (or consumed in) nutritional supplements; dietary supplements; medical foods; nutriceuticals; food-stuffs such as pharmaceutical-benefit foods (e.g., “phoods”); beverages including traditional (e.g., regular oatmeal, whole-grain breads), fortified (e.g., orange juice with calcium); and “designer” products (e.g., protein bars, smart spreads, smart bars, energy bars). The alkaline protease as described herein may be formulated in health bars, confections, animal feeds, cereals, dietary supplements, yoghurts, cereal coatings, foods, nutritive foods, functional foods, and combinations thereof.

### Dosages

The amount of active compound in the composition may vary according to factors such as the disease state, age, gender, patient history, risk factors, predisposition to disease, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

The dose range for adult human beings will depend on a number of factors including the age, gender, weight, and condition of the patient and the administration route. The dosages as suitable for this invention may be a composition, a pharmaceutical composition, or any other compositions described herein.

For each of the recited embodiments, the dose range for a patient can be 10, 20, 30, 40, 50, 60, 70, 80, 90, or 100 mg/kg and all increments therein. Preferably, the dose range for a patient can be 10-50 mg/kg and all increments therein. Alternatively, the dose range for a patient can be 25-50 mg/kg and all increments therein.

For each of the recited embodiments, the dose range for a patient can be 1, 2, 3, 4, 5, 6, 7, 8, 9, 10, 20, 30, 40., 50, 60, 70, 80, 90, 100, 200, 300, 400, 500, 600, 700, 800, 900, 1000, 1250, 1500, 1750, 2000, 2250, 2500, 2750, 3000, 3250, 3500, 3750, or 4000 mg per day and all increments therein. Preferably, the dose range for a patient is 1000 mg per day but can be 2000, 3000, or 4000 mg per day.

For each of the recited embodiments, the dose, if administered via injection (i.p.), can be 1, 2, 3, 4, 5, 6, 7, 8, 9, or 10 μg and all increments therein, per injection. Preferably, the dosage when administered via injection is 10 μg.

For each of the recited embodiments, the dosage is typically administered once, twice, or thrice a day. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage may be administered for as long as symptoms persist. The patient can require “maintenance treatment” where the patient is receiving dosages every day for months, years or the remainder of their lives. In addition, the composition of this invention may be to effect prophylaxis of recurring symptoms. For example, the dosage can be administered once or twice a day to prevent the onset of symptoms in patients at risk, especially for asymptomatic patients.

For each of the recited embodiments, the patient can receive “pretreatment” with the alkaline protease wherein the alkaline protease is administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition requiring prolonged treatment, the dosage of alkaline may be administered for as long as symptoms persist.

For administration via injection, it is preferred that the treatment begin as a course of 4 injections at 0, 12, 24, and 36 hours. The injections then can continue once, twice, or thrice a day for as long as symptoms persist. Alternatively, the injections can be maintained to prevent the recurrence (or replace) of disease. Also, the injections can be administered as a prophylaxis for patients at risk, especially asymptomatic patients.

For each of the recited embodiments, the dosage of alkaline protease may be adjusted based on specific activity, in particular, specific activity measured by cleavage of salmon protamine (nmoles Leucine/30 min./μg protein) and/or Val-leu-lys-pNA (mOD/min/μg protein). Preferably, pharmaceutical compositions of alkaline protease may comprise at least about 300, more preferably about 900 salmon protamine (nmoles Leucine/30 min./μg protein). However, pharmaceutical compositions of alkaline protease comprising at least about 30, or preferably about 100 Val-leu-lys-pNA (mOD/min/μg protein) measured at 405 nm, may be used.

The dosage can be administered as a single dose, a double dose, a triple dose, a quadruple dose, and/or a quintuple dose. The dosages can be administered singularly, simultaneously, and sequentially.

For each of the recited embodiments, the dosage of alkaline protease can be a therapeutically effective amount of alkaline protease, an amount effective for prophylaxis, and/or an amount effective for prevention. The dosage of alkaline protease can be an amount of alkaline protease effective to reduce inflammation, an amount effective to prevent inflammation, to reduce the severity of inflammation, to eliminate inflammation, to slow the development of inflammation, to prevent the development of inflammation, and/or effect prophylaxis of inflammation.

The dosage form can be any form of release known to persons of ordinary skill in the art. The preferred dosage forms include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics, and combinations thereof is known in the art.

It will be appreciated that the pharmacological activity of the compositions can be monitored using standard pharmacological models that are known in the art. Furthermore, it will be appreciated that the inventive compositions can be incorporated or encapsulated in a suitable polymer matrix or membrane for site-specific delivery, or can be functionalized with specific targeting agents capable of effecting site specific delivery. For instance, the dosage form may be made such that it preferably releases in the duodenum, the jejunum, or the ileum. These techniques, as well as other drug delivery techniques are well known in the art.

The alkaline protease can be administered in the same formulation (e.g., the same pill) or in a separate formulation as the active agent. It is preferred that the alkaline protease be co-administered with the active agent.

The alkaline protease can be administered with the active agent simultaneously, sequentially, prior to, or after administering of the active agent. Where the administration of alkaline protease is simultaneous, the alkaline protease and the active agent are administered together or within a very short time interval (e.g., 5 minutes). Where the administration of the alkaline protease is administered as pre-treatment, the alkaline protease is administered prior to administration of the active agent for any length of time contemplated herein.

### Routes of Administration

The compositions described herein may be administered in any of the following methods parenteral, intranasal, transmucosal, intravenous, intraperitoneal, intramuscular, sublingual, pulmonary, transdermal, buccal, or oral. The preferred routes of administration are buccal and oral. The administration can be local, where the composition is administered directly, close to, in the locality, near, at, about, or in the vicinity of, the site(s) of disease, e.g. inflammation or systemic, wherein the composition is given to the patient and passes through the body widely thereby reaching the site(s) of disease. Local administration can be administration to the cell, tissue, organ, and/or organ system which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral wherein the desired effect is systemic, composition is given by other routes than the digestive tract.

### EXAMPLES

The examples contained herein are offered by way of illustration and not by any way of limitation.

### Example 1

### Production of Alkaline Protease Powder

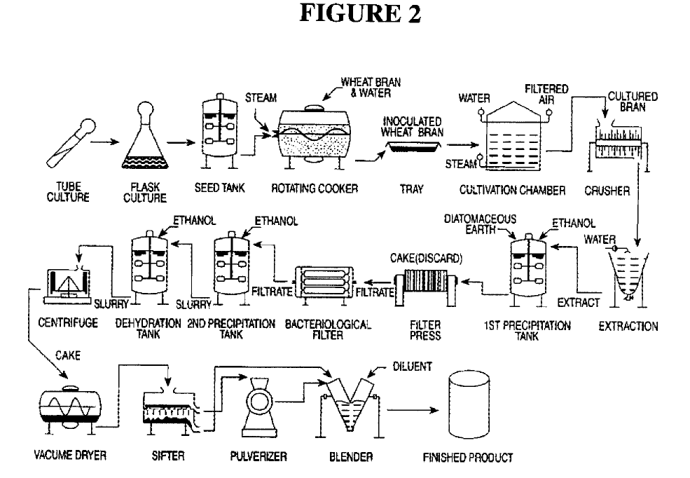

A method for producing alkaline protease from various species of Aspergillus fungi via solid state fermentation is illustrated diagrammatically in FIG. 2, although other production methods known in the art are also acceptable. Referring to FIG. 2, the fermentation process begins by taking a population of the desired fungi from a test tube culture and transferring it to a large flask for additional growth. This cultured fungi is then moved to a seed tank where it is further propagated. The resulting concentrated suspension of fungi is then transferred to a rotating cooker and mixed with sterilized koji (wheat, soy, or rice bran), water and steam where it is cooked for a sufficient period of time to inoculate the koji with fungi. The inoculated koji is then moved onto large trays, which are then transported to a cultivation chamber where the fungi are permitted to grow. Fermentation under controlled temperatures and humidity conditions may take from a few days to a week or more to complete.

At the conclusion of fermentation, the cultured koji is then transferred to a crusher device, which pulverizes the koji mash. The resulting pulverized mash is then moved to an extractor to filter the particulate matter from the slurry. For some processes, there may also be microfiltration or ultrafiltration steps to concentrate the aqueous enzymes before precipitation. The slurry is then moved to a first precipitation tank where it is mixed with ethanol and filtered through diatomaceous earth and then run through a filter press where the cake is discarded. The filtrate from the filter press is then processed through a bacteriological filter before it is moved to a second precipitation tank for further filtering and precipitation. The ethanol precipitation and bacteriological filter steps produce enzymes that are microbially very “clean” (e.g., they have very low microbes when compared to other food products such as fluid or pasteurized milk). The slurry is then centrifuged and the resulting cake is transferred to a vacuum dryer for drying. The dried proteinaceous material is then passed through a sifter and then a pulverizer to reduce the particle size. This material is then placed in a blender and diluent may or may not be added to standardize the potency of the finished powder product. This powder contains a heterogeneous mixture of microbial proteases, including some alkaline protease.

The protease powder prepared according to Example 1 is a preparation from A. oryzae is a heterogeneous preparation that cleaves salmon protamine sulfate, a common protease substrate, and inactivates mouse TNF-α by proteolysis. A commercially available version of this product, Protease 6.0, may be obtained from Amano Enzyme Co. Nagoya, Japan, for example.

TABLE 2
pH Activity Profile of Protease Powder prepared according to Example 1

| pH | Activity (%) |
|---|---|
| 3 | 25 |
| 4 | 50 |
| 5 | 75 |
| 6 | 90 |
| 7 | 100 |
| 8 | 90 |
| 9 | 85 |
| 10 | 80 |
| 11 | 25 |

### Example 2

### Purification of Alkaline Protease from the Protease Powder Prepared According to Example 1

The product of Example 1 is similar to Protease 6.0 and it may be processed as described in this Example to purify alkaline protease.

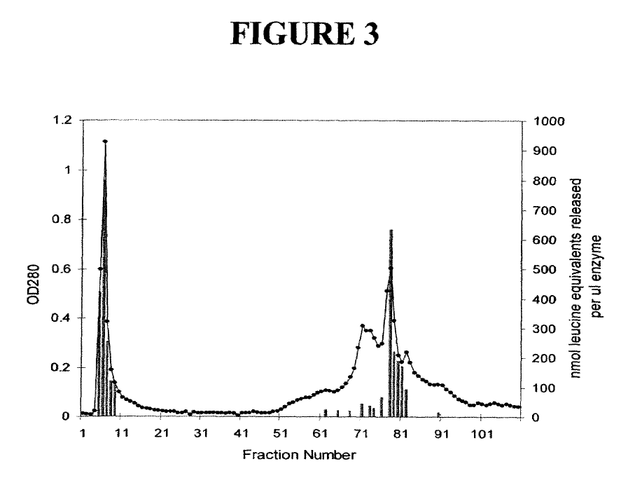

A solution of the protease powder prepared according to Example 1 was applied to the anion exchange resin Source 30Q™ (Pharmacia) at a semi-alkaline pH, and bound protein was eluted with a linear NaCl gradient. A significant portion of the protein did not bind to the ion exchange resin at pH ˜8.0 (30Q Peak I) and contained substantial proteolytic activity (FIG. 3). These unbound fractions were pooled and retained for further fractionation.

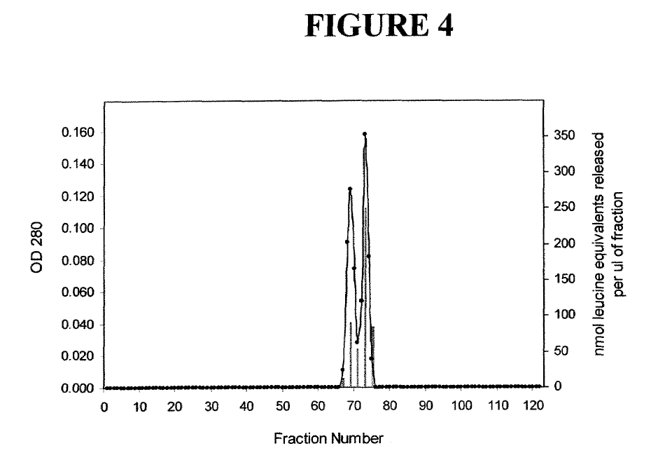

Among the bound material, a peak eluting at 250-280 mM NaCl at pH ˜8.0 also contained activity (shaded area, FIG. 3). This material was designated as “30Q Peak IV,” and gel electrophoresis contained a single dominant protein species. Peak I from 30Q chromatography was then concentrated and applied to the cation exchange resin Source 30S at pH 5.5 in acetate buffer. Two protein peaks eluted at 10-60 mM NaCl in a linear NaCl gradient (FIG. 4), and both peaks had proteolytic activity against protamine (shaded bars). Peak II from 30S was further separated by gel filtration chromatography (FPLC on Superdex 75) and eluted as a single peak with an apparent molecular mass of 29 kDa.

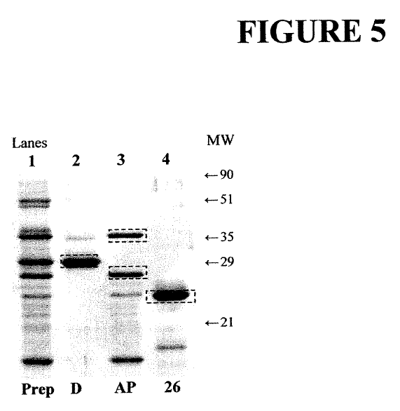

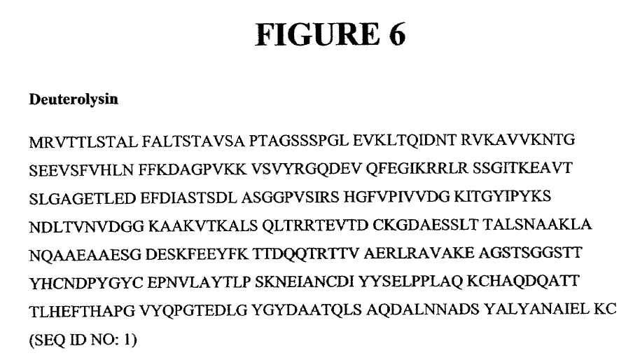

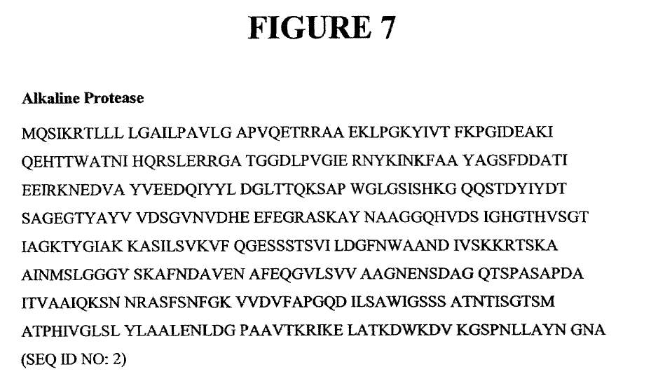

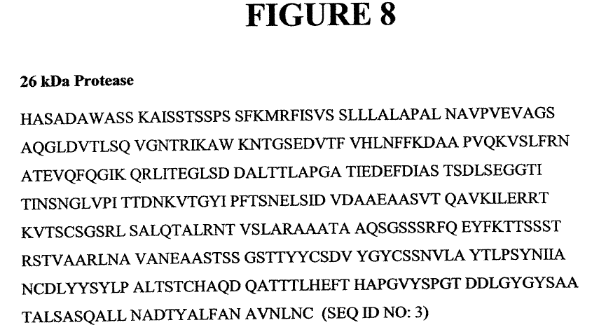

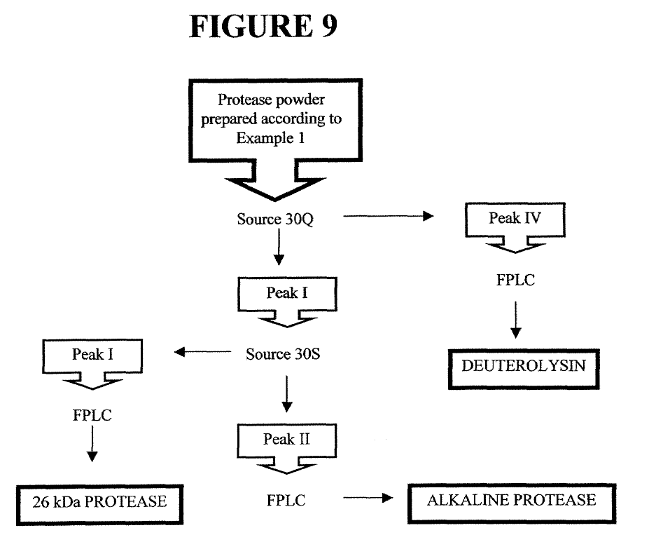

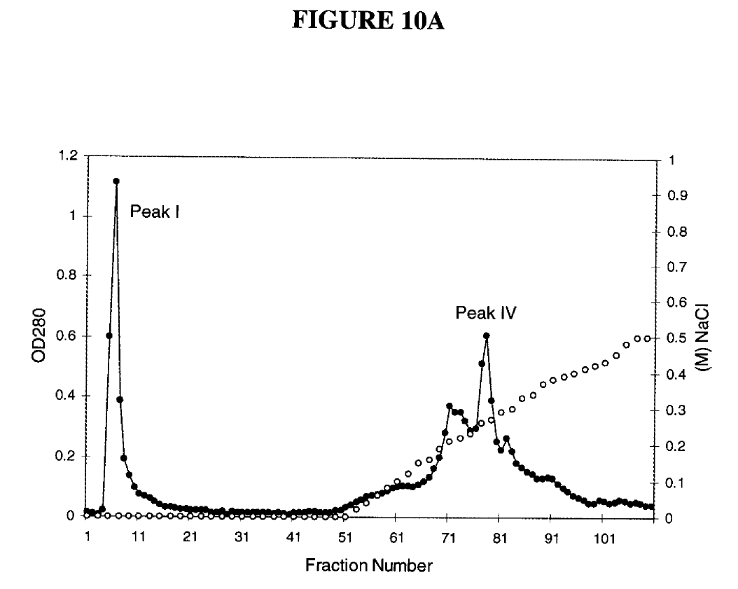

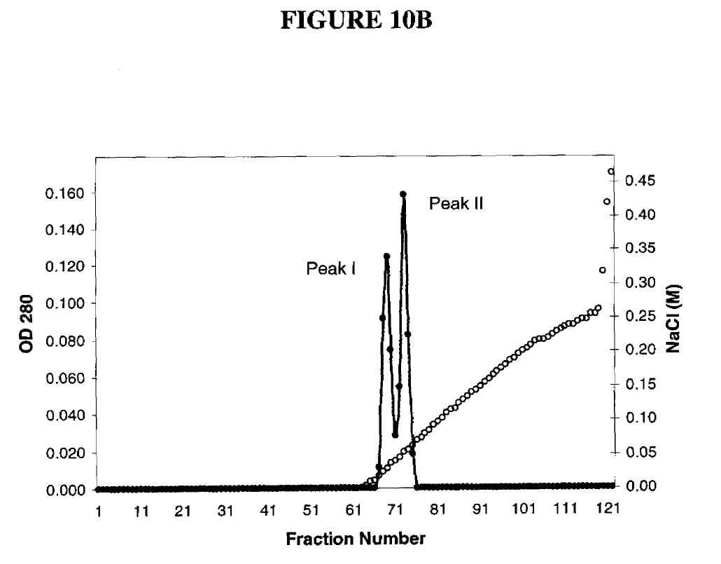

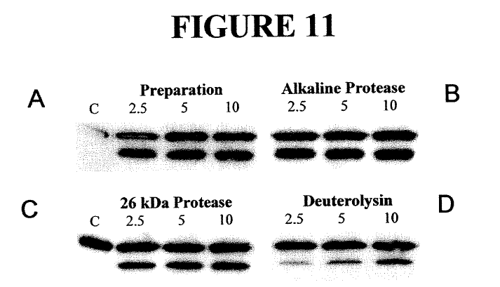

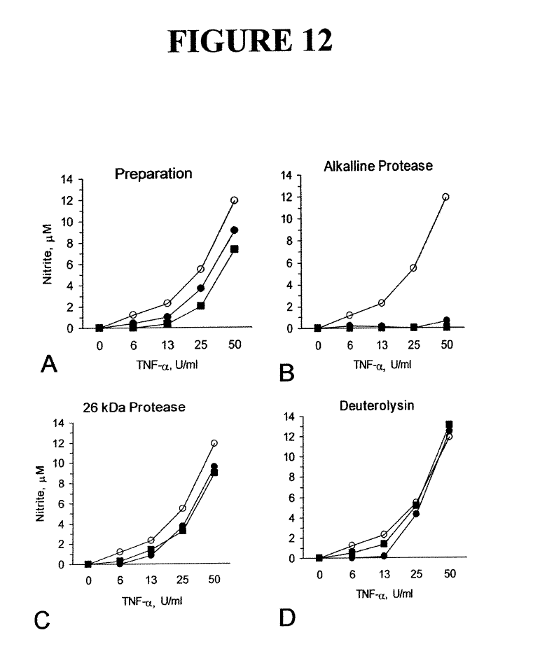

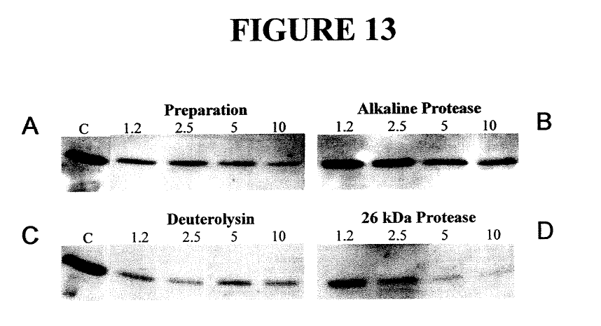

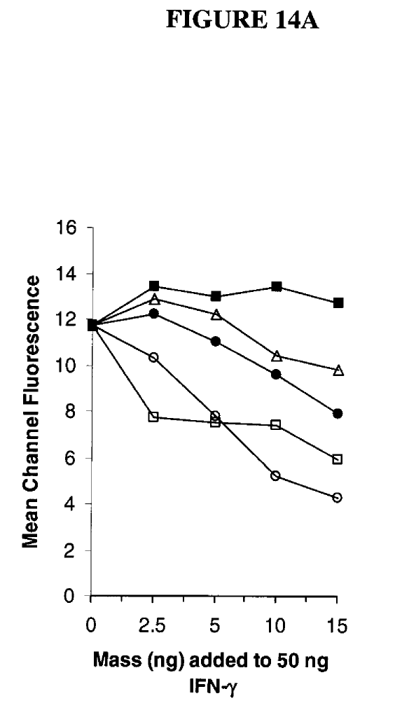

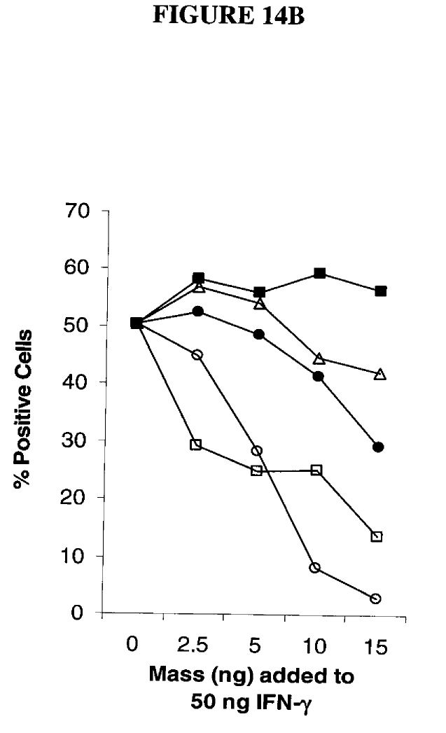

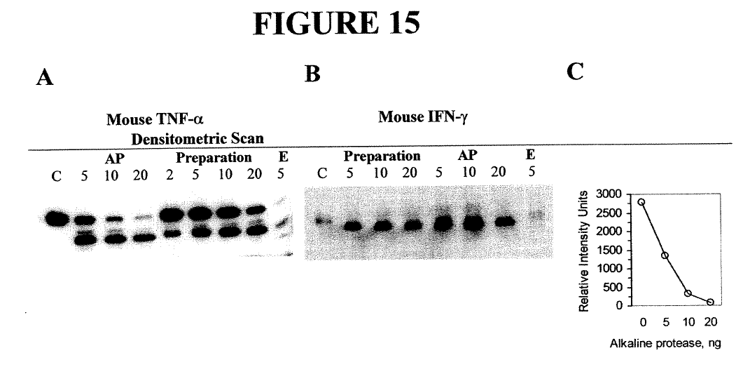

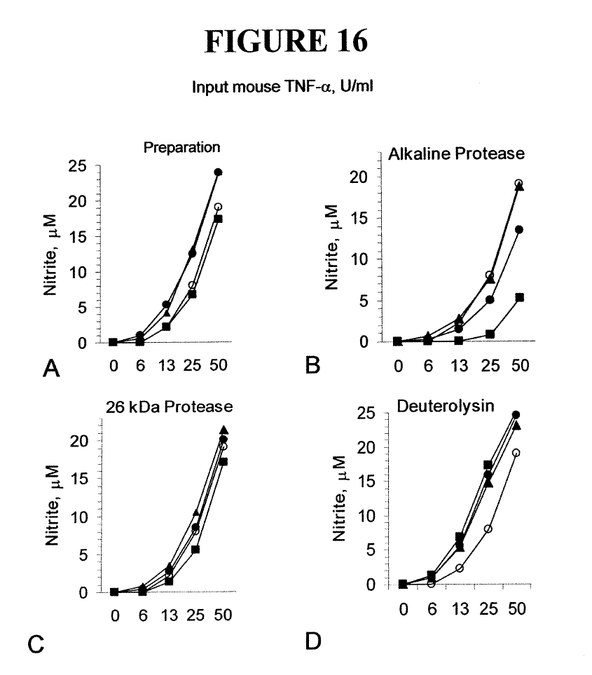

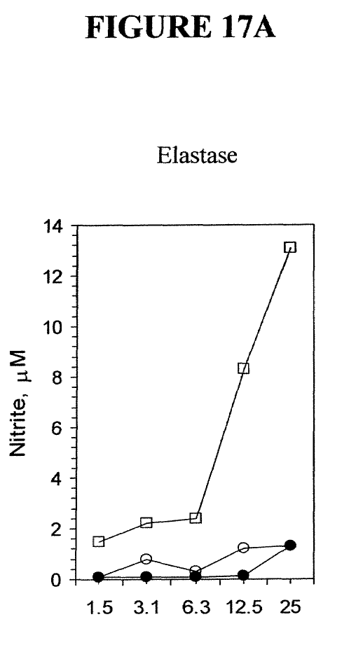

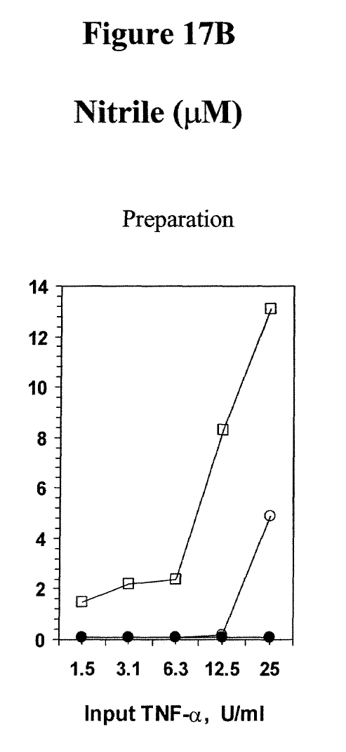

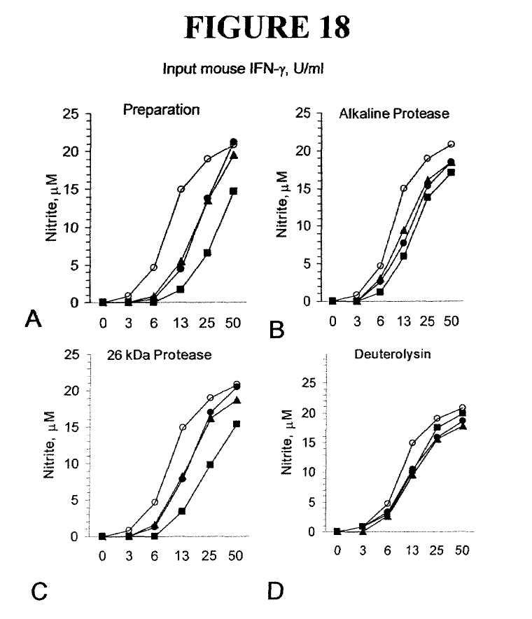

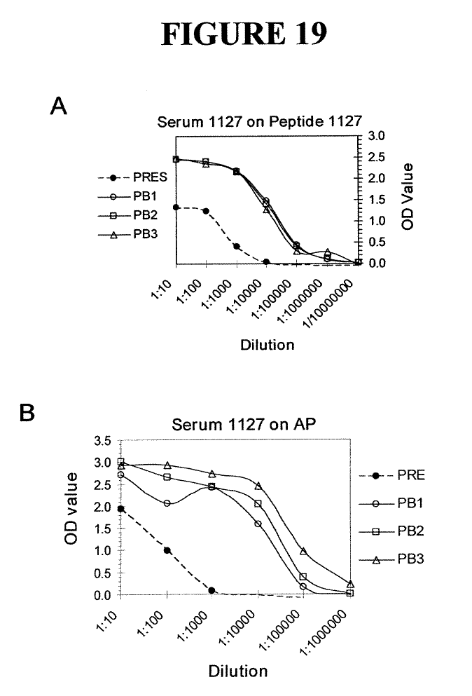

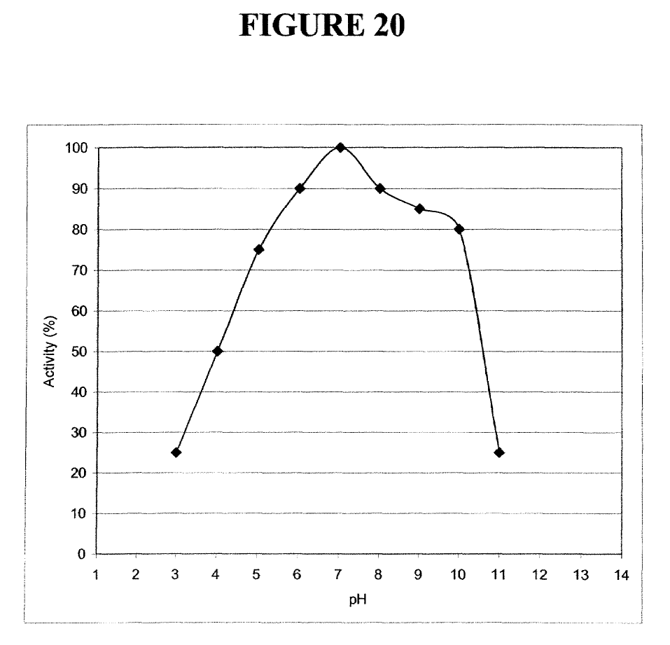

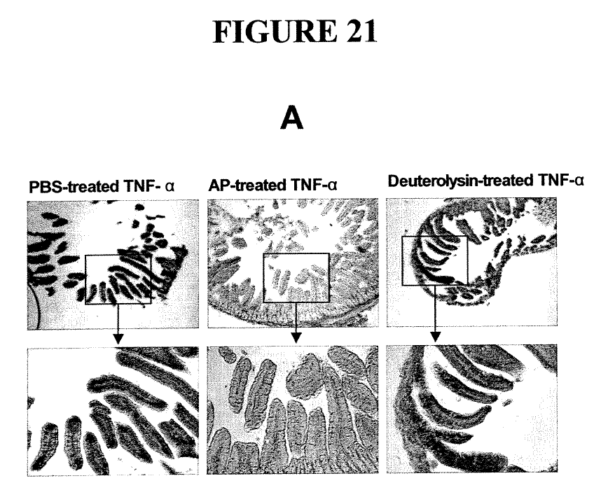

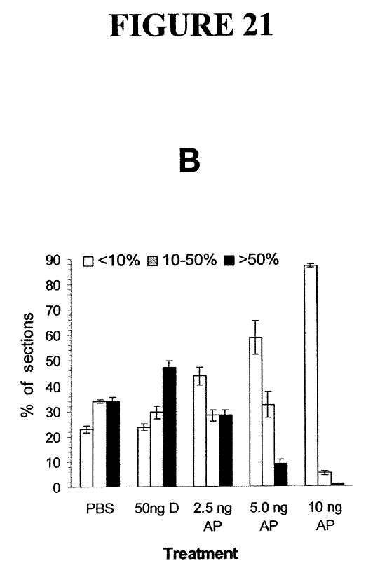

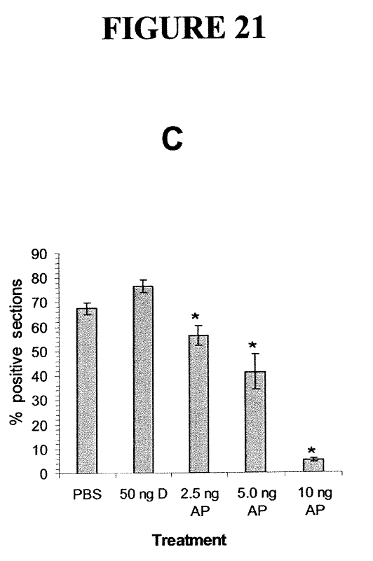

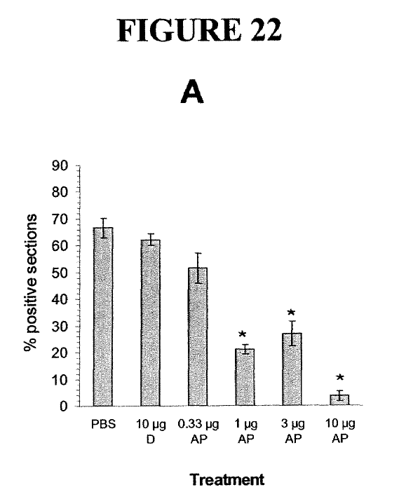

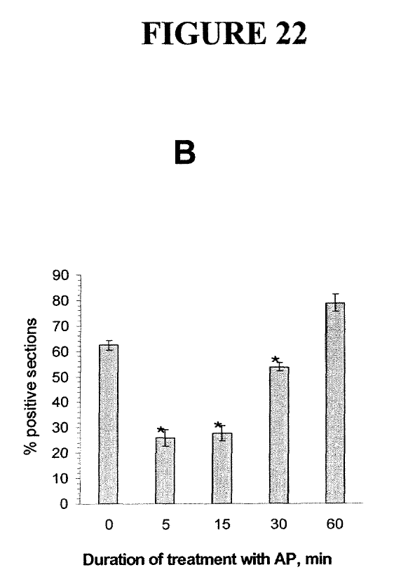

The starting material (the protease powder prepared according to Example 1) and the individual pooled fractions containing proteolytic activity were examined by sodium dodecyl sulfate-polyacrylamide gel electrophoresis (SDS-PAGE) under reducing conditions. Protein fractions were analyzed on a 12% SDS-PAGE gel and transferred either to Immobilon-P membranes for Western blot analysis with anti-AP or Immobilon-P^{SQ} (ISEQ 08100)(Millipore) for amino-terminal sequencing of selected bands. The sequencing was performed by the Molecular Biology Resource Facility at the University of Oklahoma Health Sciences Center (Oklahoma City, Okla.). The protease powder prepared according to Example 1 was found to contain no less than 20 distinct protein bands that could be stained with Coomasie Blue (Lane 1, FIG. 5). Peak IV from 30Q chromatography (lane 2) had a predominant species with an apparent molecular mass of 30 kDa. Lane 3 shows the appearance of 30S Peak II, which contained two major species (28 and 36 kDa) and a lower molecular mass peptide. Lane 4 was loaded with the first peak obtained from 30S chromatography and appeared as a single predominant species (26 kDa).

Thus, three distinct protein fractions with proteolytic activity were recovered from ion exchange chromatography and each contain unique predominant species when analyzed by SDS-PAGE. Each of the species identified in FIG. 5 by a dashed box was then recovered by SDS-PAGE followed by electrophoretic transfer to PVDF membranes. After staining the membranes, the selected protein bands were subjected to N-terminal amino acid sequencing. Note that two species from lane 3 were sequenced. The sequencing results are listed in Table 3.

TABLE 3
N-terminal amino acid sequencing of the proteases purified from the
protease powder prepared according to Example 1.

| | FIG. 4 | | | Identical |
|---|---|---|---|---|
| | lane | | N-terminal | sequence |
| Fraction | number | Apparent Mr | Sequence | match with:^{a} |
| 30Q Peak IV | 2 | 30 kDa | TEVTDCKGD | A. oryzae |
| | | | (SEQ ID NO: 4) | Deuterolysin |
| 30S Peak II | 3 | 28 and 36 kDa | GLTTQKSAP | A. oryzae |
| | | | (SEQ ID NO: 5) | Alkaline |
| | | | | Protease |
| 30S Peak I | 4 | 26 kDa | TKVTSNSGSR | A. oryzae |
| | | | (SEQ ID NO: 6) | Protease |

^{a}For the amino acid sequence of these proteins, see FIGS. 6-8.

The three proteases identified from the protease powder prepared according to Example 1 were deuterolysin (neutral protease II) (FIG. 6), alkaline protease (oryzin) (FIG. 7), and a protease cloned recently by Novozyme, labeled the “26 kDa protease.” (FIG. 8) The purified protease was identical with the Novozyme protein through 10 residues except at position 6, where an asparagine for cysteine substitution at position 6 was found. The purified 26 kDa protease sequenced also has significant sequence homology with two other proteins Novozyme has cloned from the Aspergillus species. Notice that the 28 and 36 kDa protein bands in lane 3 (alkaline protease) had the same N-terminal sequence. Alkaline protease has been cloned and has a predicted molecular mass of 29 kDa as determined by SDS-PAGE gel (Table 4).

TABLE 4
Biochemical properties of the proteases purified from the A. oryzae protease
powder prepared according to Example 1

| Property | Deuterolysin | Alkaline Protease | 26 kDa Protease |
|---|---|---|---|
| Calculated MW based | 19,017 | 29,084 | 18,528 |
| on sequence | | | |
| Apparent MW by | 29 kDa | 28 kDa; 36 kDa | 26 kDa |
| SDS-PAGE^{a} | | | |
| pI^{b} | 4.53 | 6.53 | 6.72 |
| Net charge at | −17 at pH 8.0 | +4.3 at pH 5.5 | +2.4 at pH 5.5 |
| fractionation pH^{b} | | | |
| Metal requirements | Zn, Ca, Co | — | Unknown |
| Heat stability | Stable at 90° C. for | Stable at 37° C. | Unknown |
| | 10 min | for 3 h | |
| pH optimum^{c} | pH 6-8 | pH 7-10 | 7.0 |
| Substrate specificity^{a} | Protamine > casein; | Protamine > casein, | protamine |
| | arg-val-arg-arg | hemoglobin; val-leu-lys | |
| | (SEQ ID NO: 7) | | |

^{a}Established by inventors.
^{b}This is the net charge of the protein at the pH of the buffer used to purify it to homogeneity (FIGS. 1 and 2).
^{c}pH optima vary with substrate.

Specific activity of the purified enzymes was determined by measuring hydrolysis of protamine sulfate and D-val-leu-lys-pNA. For the former, enzyme-substrate mixtures were prepared in 20 mM Tris-HCl, pH 7 or 8, and incubated for 30 min at 37° C. After precipitation with 14% trichloroacetic acid and removal of precipitated protein by centrifugation, the reaction mixtures were diluted in 500 mM sodium citrate buffer, pH 5, combined with 2% ninhydrin and boiled for 30 min. After dilution in 50% isopropanol, absorbance was measured at 570 nm wavelength, and results were referenced to leucine as a standard. Hydrolysis of D-val-leu-lys-pNA in 20 mM Tris-HCl buffer, pH 7, was determined by measuring absorbance at 405 nm wavelength every 10 sec over 10 min and calculating the change in absorbance (mOD units) per min per μg protein. Only the purified form of alkaline protease was found to cleave val-leu-lys peptide, and this protease was also slightly more active against protamine than the other enzymes (Table 5).

TABLE 5
Specific activities of the purified proteases of A. oryzae

| | | Alkaline | |
|---|---|---|---|
| Substrate | Deuterolysin | Protease | 26 kDa Protease |
| Salmon protamine | 677 | 951 | 679 |
| (nmoles Leucine/ | | | |
| 30 min./μg protein) | | | |
| Val-leu-lys-pNA | 2.3 | 116.7 | 2.1 |
| (mOD/min/μg protein) | | | |

Overall, the results of these studies indicate that at least three major fungal proteases are present in substantial quantities in the protease powder prepared according to Example 1. These differ by a number of biochemical properties, including substrate specificity, and molecular mass.

TABLE 6
Scheme for Purification of Aspergillus oryzae Protease

| | Purification Details (See FIG. 9) |
|---|---|
| 1 | The protease powder prepared according to Example 1 is dissolved and dialyzed in 30 mM |
| | Tris, pH 8.0. An aliquot is retained for determination of total protein. |
| 2 | Conditions for Source 30Q chromatography. 3 mg the protease powder prepared |
| | according to Example 1 (based on protein, not weight) is loaded per ml of Source 30Q. |
| | Loading/running buffer: 30 mM Tris, pH 8.0 Salt gradient elution: 0-500 mM NaCI in |
| | 30 mM Tris, pH 8.0 |
| 3 | Elution profile for peak I and peak IV elution corresponds with FIG. 10A. |
| 4 | Concentrate and equilibrate Peak I from 30Q in 10 mM NaAc, pH 5.5. |
| 5 | Conditions for Source 30S chromatography |
| | 150 μg Peak I of 30Q is loaded per ml of Source 30S. |
| | Loading/running buffer: 10 mM NaAc, pH 5.5 |
| | Salt gradient elution: 0-250 mM NaCl in 10 mM NaAc, pH 5.5 |
| 6 | Elution profile for peak I and peak II elution corresponds with FIG. 10B. |
| 7 | The protease may be concentrated and equilibrated in 10 mM Tris, pH 7.0 and frozen. |
| 8 | FIG. 10 shows elution profiles from Source 30Q (A) and Source 30S (B). |

### Example 3

### Alkaline Protease Purity

The purity of an Aspergillus oryzae protease composition may be determined by SDS-PAGE and its activity against protamine sulfate, CBZ-val-leu-lys-NA, CBZ-arg-val-arg-arg-NA, AZCL-casein, mouse IFN-γ, and mouse TNF-α. Additional experiments on purity may be performed using antipeptide antibodies in Western blotting. The following parameters are met by highly purified proteases.
1. Apparent homogeneity (>98% in single peak) on FPLC or equivalent resolution gel permeation chromatography.
2. Homogeneous N-terminal amino acid sequencing provided through at least 8 residues.
3. Mass spectroscopy consistent with a single protein species, corresponding to the calculated molecular mass. Chromatography elution profiles (ion exchange and gel permeation) show a single peak.

All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 4

### Protease Stability

For determining the stability of the alkaline protease in drinking water over 0-4 days, proteases can be dissolved in autoclaved tap water at a concentration of 1 mg/ml and stored at either 25° C. or 4° C. At regular intervals (every 3 hours at 0-18 hours, every 8 hours at 18-24 hours, and every 12 hours at 24-96 hours), aliquots can be ascetically removed and tested for their ability to cleave protamine sulfate using a modification of the ninhydrin assay of Rosen. Results are expressed as % residual activity measured immediately after preparation of the protease solutions. In the instant invention, the catalytic activity of the purified alkaline protease is stable with 4° C. storage for several weeks. The alkaline protease as described herein is soluble in water and stable (retention of >80% of its activity for 2 days).

### Example 5

### Alkaline Protease Activity Assay

Reagents

Protamine sulfate from salmon (Sigma, St. Louis, Mo.): 1% (10 mg/ml) solution in reaction buffer. Fushima et al use 2% and denature the protein by boiling for 30 min in 100 mM phosphate buffer, pH 7.0. (Sigma indicates that this product has a solubility limit of 10 mg/ml.)

Leucine (Sigma, St. Louis, Mo.): Prepare 10 mM stock (1.31 mg/ml) in reaction buffer and dilute to titratable range (10-100 μM) for standard curve (See Table 1; Rosen, 1967).

Buffers and Solutions

### Phosphate Buffer, 100 mM pH 7.0

### Sodium Citrate Buffer, 0.5M pH 5.0

Trichloroacetic Acid, 14% solution in water (140 g/l)
1:1 mixture of 2-propanol (isopropanol) and water.

Procedure
1. In 1.5 ml Eppendorf tubes, combine test enzyme+reaction buffer up to 200 μl. Add 200 μl of substrate (1% protamine).
2. Incubate the mixture at 37° C. (or a temperature optimal for a given enzyme). Alternatively, one can incubate for various times if a kinetic measurement is to be made.
3. Terminate the reaction by the addition of 400 μl 14% TCA, vortex mix and incubate on ice for 30 min.
4. Remove precipitated proteins by centrifugation at 10,000 rpm for 15 min at 4° C.
5. Recover 100 μl of each sample and combine with 200 μl Sodium Citrate Buffer, pH 5.0 in a 12×75 polypropylene tube. Mix well.
6. Add 100 μl of a 3% solution of Ninhydrin (Sigma Chemical, St. Louis, Mo.). Mix, cover the tubes with marbles and place in a boiling water bath for 10 min.
7. Cool the mixtures on ice and then add 1 ml of 2-propanol/water mixtures. Shake vigorously and allow cooling at room temperature.
8. Read Absorbance at 570 nm (purple color) and calculate nanomoles by reference to a standard curve prepared with leucine. Dilute samples if necessary to achieve A₅₇₀<0.8. (Color should be stable for several hours.)
9. Activity of enzyme is expressed as nanomoles of leucine equivalents released per milligram of protein (endpoint assay) or nanomoles of leucine equivalents/min/μg protein (kinetic assay).
10. Controls include the following.
   a. Buffer control (no enzyme, no substrate)
   b. No incubation control (all components mixed and then immediately precipitated with TCA)
   c. Substrate control (w/o enzyme; incubated and the TCA precipitated)
   d. Enzyme Control (w/o substrate; incubated and the TCA precipitated)

### Example 6

### Acid Protease Activity Assay

The fungal protease powder prepared according to Example 1 contains both endo-peptidase and expo-peptidase activities, as well as significant amounts of starch-saccharifying activities.

Assay Principle

This assay is based on 30 minute enzymatic hydrolysis of a hemoglobin substrate at pH 4.7 and 40° C. Unhydrolyzed substrate is precipitated with trichloracetic acid and removed by filtration. This assay is useful for determining the proteolytic activity, expressed as hemoglobin units on the on the tyrosine basis (HUT) at pH 4.7, of protease powder preparations and subsequently purified fractions thereof. In particular, this assay is useful for determining the proteolytic activity at pH 4.7, expressed in HUT, of protease powders and subsequently purified fractions derived from Aspergillus oryzae and Aspergillus niger. This assay is useful for determining the proteolytic activity at pH 4.7, expressed in HUT, for any given protease.

Assay Apparatus
1. Constant Temperature Bath (40° C.±0.1° C.)
2. Spectrophotometer capable of measuring samples at least about 275 nm
3. Standardized pH Meter

Assay Reagents and Solution

Acetate Buffer Solution

Dissolve 136 grams sodium acetate trihydrate in sufficient water to make 500 mL. Mix 25 mL of this solution with 50.0 mL of 1M acetic acid, dilute to 1000 mL with water and mix. The pH of this solution should be at least about 4.7 (±0.02).

Substrate Solution

Weigh 4.0 grams of hemoglobin into a 250 mL beaker. With continuous stirring, add 100 ml of distilled water to which 1-2 drops of antifoam have been added. Stir for 10 minutes and adjust the pH 1.0±1.7 with 0.3 N hydrochloric acid. After 10 minutes, adjust the pH to 4.7 by adding 0.5 N sodium acetate. Quantitatively transfer the solution to a 200 mL volumetric flask and dilute to volume with distilled water. This solution is stable for about 5 days at around 4° C.

Trichloracetic Acid (TCA) Solution

Dissolve 140 g of TCA in about 750 ml of water. Transfer the solution to a 1000 mL volumetric flask, dilute to volume with water, and mix thoroughly.

Sample Preparation

Dissolve an amount of the sample in the Acetate Butter Solution to produce a solution comprising between 9 and 22 HUT/mL. (This concentration will produce an absorbance reading, in the procedure below, within the preferred range of 0.2 and 0.5).

Assay Procedure
1. Pipet 10 mL of the substrate solution into a series of test tubes. It is recommended to prepare three test tubes for each sample (2 for the reaction mixture, and 1 for the enzyme blank) and 1 test tube for substrate blank. Stopper the tubes and heat in a water bath at 40° C. for about 5 minutes.
2. Prepare the reaction mixtures by adding 2.0 mL of the sample preparation to the equilibrated substrate. Begin timing the reaction the moment the solution added. Mix by vortexing, stopper, and return to the 40° C. water bath.
3. Prepare the substrate blank by adding 2.0 mL of the Acetate Buffer Solution to the substrate blank tube. Begin timing the reaction the moment the solution is added. Mix by vortexing, stopper, and return to the 40° C. water bath.
4. After 30 minutes, add 10.0 mL of the Trichloracetic Acid Solution to each tube and shake vigorously against the stopper for about 40 seconds. Allow the test tubes to cool at room temperature (by convention ˜25° C.) for 1 hour, shaking each tube against the stopper every 10-12 minutes during this period.
5. Prepare the enzyme blanks by adding 10.0 mL of the Trichloracetic Acid Solution to 10.0 mL of the equilibrated substrate and shake well for 40 seconds. To this mixture add 2.0 ml of the Sample Preparation that has been heated in the 40° C. water bath for 5 minutes. Cool at room temperature for 1 hour, shaking the test tubes at 10 to 12 minute intervals.
6. At the end of 1 hour, shake each tube vigorously, and filter through 11 cm Whatman No. 42 filter paper (or equivalent) refiltering the first half through the same filter paper.
7. Determine the absorbance of each filtrate in a 1 cm cell at 275 nm, with a suitable spectrophotometer, using the filtrate from the substrate blank to zero the instrument.
8. Correct each reading by subtracting the appropriate enzyme blank reading and record the value so obtained as AU (Arbitrary Units).

If a corrected absorbance reading between 0.2 and 0.5 is not obtained, repeat the test using more less of the Enzyme Preparation as necessary.

Standard Curve

Transfer 100.0 mg of L-Tyrosine, chromatographic-grade (or equivalent) (Sigma-Aldrich Chemical Co., St. Louis), previously dried to constant weight, to a 1000 ml volumetric flask. Dissolve in 60 ml of 0.1 N hydrochloric acid. When the L-Tyrosine is completely dissolved, dilute the solution to volume with water, and mix thoroughly. This solution contains 100 μg of tyrosine per 1.0 ml. Prepare three more dilutions from this stock solution to contain 75.0, 50.0 and 25.0 μg of tyrosine per mL. Determine the absorbances of the four solutions at 275 nm in a 1-cm cuvette on a suitable spectrophotometer in comparison to 0.006 N hydrochloric acid.

Prepare a plot of absorbance per μg of tyrosine. Multiply this value by 1.10, and record it as “As”. A value of approximately 0.0084 should be obtained.

Calculations

HUT/g=((Au/As)×22/30 w)

where:
Au=Absorbance of enzyme filtrate at 275 nm
As=Slope of the curve of Abs.-vs.-μg Tyrosine×1.10
W=Weight, in grams, of enzyme added in the 2.0 ml aliquot
22=Final volume of the test solution: 30=Reaction time (minutes)

### Example 7

### Cleavage and Inactivation of Human Cytokines by Three Aspergillus oryzae Proteases

### Human TNFα Hydrolysis

Human TNF-α is cleaved and inactivated by Aspergillus oryzae alkaline protease. Purified cytokine (50 ng) was first combined with the following amounts [2.5, 5, and 10 ng] of protease powder prepared according to Example 1 (preparation), as well as alkaline protease, 26 kDa protease, and deuterolysin prepared according to Example 2. Each mixture was incubated overnight at 37° C. and examined by Western blotting for evidence of proteolysis. (FIG. 11A-D) The protease powder produced according to Example 1 cleaved human TNF-α to produce a fragment approximately 1-2 kDa smaller than the native cytokine. Alkaline protease and, to a lesser extent, 26 kDa protease each produced a similar pattern of cleavage, while deuterolysin was without significant effect. Because the antibody used in this Western blot was specific for the carboxyl terminus of human TNF-α, these enzymes cleaved the cytokine at the amino terminus yielding a smaller peptide that retained the C-terminal epitope.

### Human TNF-α Activity

Protease-treated human TNF-α was then tested for its bioactivity in the C2C12 cell assay. The results are shown in FIG. 12 and indicate that A. oryzae alkaline protease selectively inactivated human TNF-α, while the other proteases had only a limited effect. Although the 26 kDa protease partially digested human TNF-α, it did not significantly alter the biological activity of the cytokine. (FIG. 12A-D)

### Human IFN-γ Degradation

While alkaline protease is active against human TNF-α, the protease did not cleave human IFN-γ. (FIG. 18 A-D) By contrast, both deuterolysin and 26 kDa protease cleave human IFN-γ. Cleavage was evidenced by the disappearance of the native 17 kDa IFN-γ band, rather than the appearance of distinct cleavage peptides. Because the antibody used in this Western blot was specific for the carboxyl terminus of human IFN-γ, these enzymes attack the cytokine at the C terminus (e.g., there is a loss in reactivity with the anti-cytokine antibody, rather than the appearance of smaller peptide products).

FIG. 14 A-B shows the effects of the purified A. oryzae proteases on the Fc receptor-inducing activity of human IFN-γ. Samples of the cytokine were first treated with the indicated Pseudomonas or fungal proteases: Elastase (∘); Preparation (); Alkaline Protease (□); Deuterolysin (▪); and 26 kDa Protease (Δ). The treated samples were added to human promyelocytic U937 cells to induce expression of Fc receptors for IgG, which was detected by flow cytometry. The results are plotted either as a decrease in of mean channel fluorescence (A) or a decrease in % positive cells (B) plotted against increasing protease amounts.

### Example 8

### Cleavage and Inactivation of Mouse Cytokines by Purified Aspergillus oryzae Alkaline Protease, Deuterolysin, and 26 kDa Protease

### Hydrolysis of Murine TNF-α

The protease powder prepared according to Example 1 inactivates mouse recombinant TNF-α by limited proteolysis. (FIG. 16 A-B) This property was established by measuring the loss of activity of TNF-α in a bioassay in which the cytokine was used to coactivate mouse myoblast C2C12 cells for the production of NO by transcriptional activation of the inducible NO synthase (iNOS) gene. The assay can be used to measure either TNF-α or IFN-γ activity by adding test samples containing one of the cytokines to cells stimulated with an excess of the other cytokine. Nitrite is a stable endproduct of NO produced by the cells. Using Pseudomonas elastase as a positive control, these data show that the protease powder prepared according to Example 1 substantially reduced the bioactivity of TNF-α in this assay. (FIG. 16A-B).

The protease powder prepared according to Example 1 also caused a demonstrable cleavage of mouse TNF-α as revealed by Western blotting. (FIG. 15 A-C) In this experiment, the protease powder prepared according to Example 1 also cleaved both mouse TNF-α and mouse IFN-γ yielding products with molecular masses only slightly less than those of the native cytokines. These principal peptide products were surprisingly resistant to further proteolytic cleavage. Of interest, alkaline protease (AP) purified from the protease powder prepared according to Example 1 evidenced the same substrate specificity. Because the anti-TNF-α used here was specific for an epitope located in the carboxyl terminus of the cytokine, the pattern of cleavage is most consistent with attack at the amino terminus of TNF-α (e.g., there is a loss of molecular mass without a loss of the epitope recognized by the antibody).

### Murine TNF-α Activity

The three purified proteases were then compared for their ability to inactivate mouse TNF-α using the C2C12 cell bioassay. Two principal findings are apparent from this analysis, which are shown in FIG. 17 A-B. First, alkaline protease showed a dose-dependent inactivation of mouse TNF-α as reflected by the reduced ability of protease-treated TNF-α to co-activate C2C12 cells for NO (nitrite) production. While the 26 kDa protease appeared to be devoid of such activity, deuterolysin actually enhanced NO production slightly.

### Murine IFN-γ

To evaluate the specificity of this effect, the three Aspergillus oryzae proteases were tested for their ability to inactivate mouse IFN-γ, and a different pattern was seen. As shown in FIG. 18, the protease powder prepared in according to Example 1 showed some activity against the cytokine, and this was primarily associated with the 26 kDa protease. Alkaline protease did not significantly affect the activity of mouse IFN-γ. This was an unexpected finding given the ability of alkaline protease to cause partial proteolysis of IFN-γ. (FIG. 18) Thus, alkaline protease partial degrades mouse IFN-γ without altering its bioactivity. These data demonstrate that the effects of alkaline protease on mouse TNF-α are specific for this cytokine and not seen when mouse IFN-γ was exposed to the enzyme.

Table 7 summarizes the results of these studies on human and mouse cytokines and suggests the following conclusions: (i) alkaline protease is the active component of the protease powder prepared according to Example 1 preparation that targets both mouse and human TNF-α; (ii) Alkaline protease is active against human IFN-γ and partially hydrolyzes mouse IFN-γ (FIG. 15 A-C), but does not inactivate the mouse cytokine; (iii) mouse IFN-γ is somewhat susceptible to inactivation by 26 kDa protease (FIG. 18), although the protease does not inactivate human IFN-γ (FIG. 13).

These results indicate that the purified proteases of A. oryzae show both species and substrate specificity and, importantly, can inactivate an important proinflammatory cytokine, TNF-α. Therefore alkaline protease is potent anti-inflammatory agent with therapeutic utilities.

TABLE 7
Summary of the effects of A. oryzae Proteases on Mouse and Human
Cytokines

| | Human | Human | | |
|---|---|---|---|---|
| Enzyme | TNF-α | IFN-γ | Mouse TNF-α | Mouse IFN-γ |
| The protease | + | + | + | +/− |
| powder prepared | | | | |
| according to | | | | |
| Example 1 | | | | |
| Alkaline Protease | + | + | + | −^{a} |
| Deuterolysin | − | − | − | − |
| 26 kDa Protease | −^{a} | − | − | +/− |

+ Indicates that the protease inactivated the cytokine.
^{a}Cleavage of the cytokine occurred without inactivation.

### Example 9

### Effects of AP on TNF-α-Initiated Inflammation In Vivo

TNF-α is a potent mediator of cell death in vivo and has been shown to cause apoptosis in intestinal mucosal epithelial cells when administered systemically. A series of experiments was performed in which the activation of caspase-3 within the intestine was measured. Preliminary experiments showed that 5-10 ng of TNF-α injected i.p. was sufficient to induce the expression of activated caspase-3 in the villous epithelium of the duodenum. To determine whether AP blocked TNF-α activity in this in vivo assay, the cytokine was first treated either with PBS, AP or D for 2 h, the reaction mixtures were inactivated by the addition of FBS, and the cytokine-protease mixtures were injected i.p. into mice. One hour later the mice were euthanized, samples of their intestines were collected and fixed, and tissue sections were analyzed by immunohistology for the expression of activated caspase-3 (FIG. 21-22). The results are shown in FIG. 22 in which data are expressed in two forms, either as the frequency distributions of sections expressing different levels of activated caspase-3 (FIG. 21B) or as the percentages of tissue sections in which 10% or more of the villi stained for activated caspase-3 (FIG. 21 C). Treating TNF-α with increasing concentrations of AP, but not D, destroyed the ability of the cytokine to activate caspase-3 in intestinal epithelial cells.

A modification of this experiment was then performed by injecting various amounts of the proteases for 15 min prior to challenge with TNF-α (FIG. 22). Intestinal tissues were again recovered 1 h later and the expression of activated caspase-3 was measured. Pretreating mice with AP at doses of 1-10 μg blocked the action of TNF-α in vivo, whereas pretreatment with D was without a significant inhibitory effect (FIG. 22A). However, the ability of AP to provide protection against the apoptotic effects of TNF-α in vivo was limited in its duration (FIG. 22B). Thus, pretreatment of the mice for up to 30 min prior to TNF-α challenge prevented caspase-3 activation, whereas delaying cytokine challenge beyond 30 min diminished the protective effect of the protease.

Mice were injected i.p. with 5 ng of recombinant mouse TNF-α or protease-treated TNF-α, and 2 cm segments of their duodenums were collected 1 h later. The tissues were fixed with 4% paraformaldehyde and 5 μm thick sections were prepared and stained for activated caspase-3 by immunoperoxidase techniques as known in the art. The sections were first treated with rabbit antibody to activated (cleaved) mouse caspase-3, and bound antibody was detected using a Histostain-SP kit. For each mouse, tissues were scored by enumerating the number of sections in which less than 10%, 10-50%, or >50% of the intestinal villi expressed activated caspase-3. At least 48 sections were analyzed in this fashion from each mouse.

### Example 10

### Characterization of Antibodies to Aspergillus oryzae Alkaline Protease

Using the amino acid sequence of alkaline protease, a peptide mimicking the N-terminus of the protease was produced and used to raise a rabbit antibody to the peptide (Serum 1127): residues 48-56 of SEQ ID NO: 2: HEEFEGRAS (SEQ ID NO: 8) and residues 245-253 of SEQ ID NO: 2: KRIKELATK (SEQ ID NO: 9).

The reactivity of the antibody in ELISA against the peptide and alkaline protease is shown in FIG. 19A-B. These anti-protease antibodies are useful for characterizing the purity of large-scale preparations of the enzymes and for the immunological localization of the enzymes in tissues and subcellular compartments following in vivo delivery. These antibodies can also be used to detect the protease in body tissues and fluids using standard immunological techniques. All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

Antibodies suitable for use in this invention may be monoclonal, polyclonal, single-chain, humanized, and/or chimeric. Antibodies may also be made recombinantly or through use of Xenomouse™ technology. Usually, mice are used to make monoclonal antibodies and rabbits are used to make polyclonal antibodies using methods known in the art which many of which are described in Ed Harlow & David Lane (Eds) (2^{nd} Ed.) (1999) Antibodies: A Laboratory Manual Cold Spring Harbor Laboratory Press, New York, which is incorporated by reference.

Further, antibodies described herein include but are not limited to, monoclonal, multispecific, human or chimeric antibodies, single chain antibodies, Fab fragments, F(ab′) fragments, anti-idiotypic (anti-Id) antibodies (e.g., anti-Id antibodies to antibodies of the invention), intracellularly-made antibodies (e.g., intrabodies), and epitope-binding fragments thereof. The immunoglobulin molecules of the invention can be of any isotype (e.g., IgG, IgE, IgM, IgD, IgA, and IgY), class (e.g., IgG₁, IgG₂, IgG₃, IgG₄, IgA₁, and IgA₂) or subclass of immunoglobulin molecule. Preferably, an antibody of the invention comprises, or alternatively consists of, a VH domain, VH CDR, VL domain, or VL CDR having an amino acid sequence of any one of those referred to in Table 1, or a fragment or variant thereof. Immunoglobulins may have both a heavy and light chain. An array of IgG, IgE, IgM, IgD, IgA, and IgY heavy chains may be paired with a light chain of the kappa or lambda forms.

These anti-alkaline protease antibodies are useful for the characterizing the purity of large-scale preparations of the enzymes and for the immunological localization of the enzymes in tissues and subcellular compartments following in vivo delivery.

### Example 11

### Detection of Active Alkaline Protease

Translocation of alkaline protease across polarized epithelial cell monolayers in vitro, or uptake and transport by the intestinal mucosal epithelium in vivo, can be measured. The organ, tissue, and cellular distribution of the administered alkaline protease can be monitored and assessed by the specific activity of alkaline protease found to interact with cells and tissues of the gastrointestinal tract. Immunological techniques for monitoring the fate and tissue distribution of exogenously administered alkaline protease can provide optimum sensitivity and specificity and can be designed in such a way that intact enzymes can be distinguished from partially degraded alkaline protease. For example, separate antibodies specific for the N and C termini of alkaline protease allow for the design of a capture ELISA capable of detecting undegraded enzyme.

Using the antipeptide antibodies produced against the A. oryzae proteases, such as those described in Example 10, capture ELISAs can be designed. Each anti-protease antibody can also be tested for its ability to inhibit the catalytic activity of the proteases using the protamine protease assay. These antibodies can be combined with chromogenic or fluorogenic substrates to measure the presence of enzymatically active proteases in test samples (e.g., serum or tissue extracts).

Pairs of anti-TNF-α antibodies (N and C terminus-specific) in capture ELISA systems can be used to measure pg/ml quantities of TNF-α in mouse serum and tissue culture supernatant fluids. The detection methods for ELISA are well-known in the art and include but are not limited to fluorogenic, radioactive, colormetric, and enzymatic. Controls are included to assure that the proteases do not degrade the capture or detection antibodies or the HRP conjugate (e.g., AEBSF-inactivated proteases). AEBSF (10 μM) (Sigma, St. Louis, Mo.) treatment of the proteases will destroy their catalytic activity and preclude reaction with endogenous protease inhibitors (e.g., serpins). ELISA data can be expressed as pg/ml or pg/μg of protein of sample. These sensitive immunoassays routinely measure proteins at concentrations as low as 1 pM (e.g., 30 pg/ml for a 30 kDa protein). The specificity of all positive reactions can be confirmed by repeating the reactions in the presence of 100-1000-fold molar excess of the synthetic peptide used as the immunogen.

Tissues from protease-challenged mice can be fixed in 4% paraformaldehyde and embedded in paraffin, for instance, or fixed using methods known in the art. Standard immunoperoxidase techniques can be used to detect proteases within the tissues using biotinylated forms of the protease-specific antibodies. Alternatively, cell monolayers can be grown in chamber slides to confluence and exposed to proteases. Monolayers are then to be washed with PBS and fixed with 4% paraformaldehyde, for instance, or fixed using methods known in the art. Immunocytochemistry and immunoflourescence methods are well-known in the art and can be used, such as streptavidin-conjugated HRP and diaminobenzidine (DAB). To determine whether any proteases detected in monolayers represent intact enzymes, two-color immunofluorescence can be employed, for instance, as described by Naranatt et al. (July 2002) “Characterization of γ2-human herpesvirus-8 glycoproteins gH and gL.” Arch Virol. 147: 1349-70, which is incorporated by reference. Controls can include sections or monolayers preincubated with blocking concentrations of homologous or heterologous peptides added prior to the primary antibodies.

The capture ELISA can provide a sensitive and specific assay for proteases using high-titered. Alternatively, the specific antibodies can be positively selected by peptide affinity chromatography. Protein antigens can routinely be measured in the 5-10 pg/mL range in mouse serum using these assays. Another advantage of these techniques is that they can detect antigens that are complexed with binding proteins, such as protease inhibitors. Indeed, the two-antibody ELISA approach can be modified to characterize such complexes by using an anti-protease capture antibody and a detection antibody specific for the protease inhibitor (e.g., anti-α₂-macroglobulin). This system can also be modified to measure the residual catalytic activities of captured proteases. See Castell et al. (1997) “Intestinal absorption of undegraded protesin in men: presence of bromelain in plasma after oral intake.” Amer J. Physiol. 273: G139-46, which is incorporated by reference, for the detection of bromelain. Immunohistological and immunocytological techniques may also show nonspecific staining, but there are a number of approaches to minimize these background problems. These are typically associated with endogenous cellular peroxidases, nonspecific binding sites, biotin/avidin-binding proteins or true cross-reacting antigens. The production of high-titered antibodies and the removal of unwanted reactivities by affinity chromatography can further assure specificity. All of these methods are well-known to those of ordinary skill in the art and many are described in Ausubel et al. (Eds.) (2002) Short Protocols in Molecular Biology (5th Ed.) John Wiley & Sons, Inc., which is incorporated by reference, and Harris et al. (Eds.) (2006) Cell Biology Protocols John Wiley & Sons, Inc., which is incorporated by reference.

A protease capture assay modified from Castell et al. can be used to detect catalytically active proteases. One can measure the enzymatic activity of alkaline protease at a detection limit of 30 ng using a kinetic microtiter plate assay. The assay measures a cleavage of the chromogenic peptide substrate (e.g., CBZ-val-leu-lys-p-nitroanaline) by monitoring the increase in 405 nm absorbance over time (ΔmOD units/min/μg of protein). Microtiter plates are coated with a capture anti-protease antibody that does not inhibit catalytic activity and test samples are added to the wells. After incubation overnight at 4° C., the nitroanaline peptide substrate (670 μM) is added and the change in A₄₀₅ is measured at 30 sec intervals over 30 min. The following chromogenic substrates can be used for each enzyme: CBZ-val-leu-lys-NA (Sigma) for alkaline protease (Table 4); CBZ-arg-val-arg-arg-NA (Peptide Institute) for deuterolysin; and AZCL-casein (Sigma) for 26 kDa protease (Megazyme).

### Example 12

Alkaline Protease Translocates Across Intact Monolayers of Polarized Mouse Intestinal Epithelial Cells In Vitro.

One art-accepted in vitro model system for studying transepithelial transport is the transwell system, establishing an intact polarized epithelium with immortalized mucosal epithelial cell lines on a porous filter. This culture system can characterize either apical-to-basalateral or transverse movement of molecules. A number of epithelial cell lines have been used in the art, including but not limited to human colonic Caco-2 cells, human lung epithelial A549 cells, human intestinal epithelial Int 407 cells, rat intestinal epithelial IEC6 cells, and primary rodent epithelial cells. Establishment of a continuous monolayer is verified by measuring transepithelial conductance, although some cell lines can form intact monolayers without producing significant changes in electrical potential. A variety of techniques have been used in the art to follow protein transport across epithelial monolayers, including labeling the protein with radioisotopes, fluorescent markers, or enzymatic tracers. Transwell models of epithelial transport can be used to assess the proteolytic activity in alkaline protease uptake and transport and to characterize the quantities of the alkaline protease that are translocated to the basolateral surface of the epithelial cell monolayers. Likewise, the approach can identify variables (e.g., protease-induced damage to enterocytes) that can aid in oral delivery of the alkaline protease in patients, including animals.

The human colorectal adenocarcinoma cell line Caco-2 is widely used in the art in drug screening as a predictor of intestinal epithelial transport efficiency. A number of commercially available tissue culture systems are available for this purpose, including the Multiscreen Caco-2 Assay System (Millipore) and the TRANSWELL™ System (Corning). Caco-2 cells readily form polarized enterocyte monolayers with distinct brush borders, junctional complexes and a number of active transport mechanisms (e.g., di-peptide transporters and P-glycoproteins) in vitro.

Permeability to macromolecules is readily determined quantitatively and can be used to study modulation of epithelial permeability by extracellular macromolecules, including cytokines. Transepithelial electrical resistance (TER; and its inverse conductance) is determined by the Ohmic relation and has been used as an index of barrier permeability. McKay & Baird (1999) “Cytokine regulation of epithelial permeability and ion transport.” Gut 44: 283-289.

Immortalized lines of intestinal epithelial cells, such as Caco-2 cells, are routinely employed as polarized cell monolayers in transwell systems and can be used as an in vitro model of intestinal epithelial transport. The integrity of continuous Caco-2 monolayers displaying intact tight junctions and cellular polarization are determined by measuring transepithelial electrical resistance (TER). The fate of alkaline proteases added to the apical epithelial surface can be monitored by a combination of immunological and enzymatic techniques, by examining the content of the apical and basolateral chambers of transwells and immunostaining of the cell monolayer.

Caco-2 cells (available from ATCC, Manassas, Va.) are grown to confluence in Multiscreen Caco-2 plates (Millipore). Medium is changed daily until confluence is reached and transepithelial electrical resistance (TER)>100 Ω per cm² (indicative of monolayer continuity and tight junction formation) is achieved. TER can be measured for each well of 96 well plates with a Millicell ERS probe and ohm meter. Alternatively, the exclusion of the fluorescent dye Lucifer yellow (LY) (Sigma) from the acceptor (basolateral) chamber can be measured on control wells.

Prior to undertaking studies on the translocation of proteases across epithelial cell monolayers, the potential toxicity of the alkaline protease for Caco-2 cells is shown determined using techniques known in the art. Control proteins may include bovine serum albumin (BSA) and proteases inactivated with 4-(2-aminoethyl) benzenesulfonyl fluoride hydrochloride (AEBSF) (Sigma). The limulus amoebocyte lysate (LAL) assay can be used to measure the concentration of endotoxin that contaminate each protein preparation and could alter responses to the proteases or induce apoptosis. At various times following addition of the proteases (0-48 h), tests of viability can be conducted, which may include mitochondrial dehydrogenase activity (MTT dye reduction) and staining for pre-apoptotic changes in plasma membrane integrity with propidium iodide (PI) and FITC-conjugated Annexin V (BD Pharmingen). Staining can be determined by immunofluorescence microscopy. Damage to the Caco-2 cell line from exposure to proteases can also be determined for each well by measuring lactate dehydrogenase (LDH) release in apical and basolateral chamber fluids using a kinetic assay kit (Sigma) referenced to an LDH standard. Concentrations of the alkaline proteases and exposure times can then be selected to minimize cell death.

After Caco-2 monolayers have reached confluence in dual chamber transwell assay plates, the culture medium can be replaced with Krebs-Ringer bicarbonate medium (KRB), and initial TER may be measured after a one hour stabilization period. Then different donor chambers receive the following test compounds and controls: (i) KRB; (ii) 0.01% fluorescein-Na as a hydrophilic paracellular transport marker; (iii) three-fold dilutions of each of the A. oryzae proteases in the range of 0.1-10 μM concentrations (3-300 μg/ml for alkaline protease); (iv) selected concentrations of inactivated (AEBSF-treated) (Sigma) Aspergillus oryzae proteases; (v) trypsin (1-20 FIP units/ml; Sigma); (vi) chymotrypsin (Sigma); or (vii) horseradish peroxidase (HR-P)(0.1-1 mg/ml; Sigma). HRP may serve here as a nonprotease control enzyme that is normally excluded by the monolayer. At 20 minute intervals up to 180 min, 50 μl samples should be taken from the donor (apical) and acceptor (basolateral) compartments and frozen at −80° C. until assay. Fresh KRB is added back to the chambers, and calculations of the concentrations of test proteins can be adjusted for this add-back dilution. At the end of the period of protease exposure, test samples can be removed and replaced with KRB, and cell monolayer TER is measured for each well 10 and 60 minutes later. To measure changes in paracelluar transport, fluorescence of samples from wells receiving fluorescein-Na are measured on a microplate fluorescence reader at 485 nm excitation/530 nm emission wavelengths. The HRP activity of the samples and HRP standards (Sigma) are measured using a kinetic plate assay with H₂O₂ and the chromogenic substrate TMB (Pharmingen). Protease translocation from donor to acceptor compartments may be determined by known enzymatic and immunological assays. The sensitivities of the ELISA is in the range of 1-10 pM (30-300 pg/ml for alkaline protease), e.g., one millionth the concentration in the apical chamber. One can detect catalytic activity of translocated proteases by first capturing them on microtiter plate wells; because the lower limit of detectability of alkaline protease in the chromogenic protease assay is 30 ng, one should be able to detect translocation of 0.01% of the protease added to the apical cell surface.

Conformation of molecular mass can be performed by Western blotting after immunopreciptation of the proteases from the donor and acceptor chambers with immobilized anti-protease antibodies.

Quantitative measurements of protein translocation can be made by calculating apparent permeability rates (Pₐₚₚ)(cm/sec) by the following formula:

$${P}_{app} = ( \frac{{V}_{acceptor}}{Area ( Time )} ) \times ( \frac{{[ Protease ]}_{acceptor}}{{[ Protease ]}_{initial , donor}} )$$

where V_{acceptor} is the volume (ml) of the acceptor well, Area is the surface area of the membrane (0.11 cm²), Time is the total transport time in min and the protease concentrations are those of the acceptor well at the end of incubation and the initial donor well.

Immunocytological techniques can provide the initial methods for determining the cellular localization of alkaline proteases with the Caco-2 monolayers. For analysis of cell structure, immunofluorescence staining for myosin and the ZO-1 tight junction protein can also be performed using specific antibodies (Amersham). Co-localization studies can employ labeled antibodies which are well known in the art. Alternatively, confocal microscopy may be used. All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 13

### Distribution of Alkaline Proteases within Mouse Tissues Following Administration by Several Routes, Including the Oral Route

Alkaline protease, when administered by the oral or intragastric routes, can be subsequently found associated with the intestinal mucosal epithelium and extra-intestinal tissues.

Mice can be administered a given alkaline protease by one of the following routes: (i) as a single dose by intragastric intubation; (ii) ad libitum in their drinking water; (iii) as a single dose injected by the intraperitoneal route; or (iv) as a single intravenous injection. The intestinal and extra-intestinal distribution of the proteases can be determined using immunohistological techniques, and dose- and time-dependent effects can be determined.

Groups of 3 CF1 mice each can be given a single dose (0.25 ml) of the protease (5, 10 or 20 mg/kg body weight; e.g., 125, 250 or 500 μg per 25 g mouse) by the intragastric route. Controls can receive distilled water or equivalent concentrations of ovalbumin (OVA). Proteases are usually administered to mice with the aid of a blunt ended stainless steel feeding needle. Two, four, eight and twelve hours later the following tissues can be recovered and fixed, especially segments of duodenum, jejunum, ileum and proximal colon, liver, mesenteric lymph nodes, kidney, spleen, and lungs. For each mouse, a set of each tissue can be embedded in paraffin, sectioned and mounted together on a single microscope slide. Care should be taken in recovering intestinal segments to include samples with Peyer's patches. Blood may be collected for serum at the time of euthanasia. Immunoperoxidase staining of the tissues is performed as described in the art using peptide-specific anti-alkaline protease, anti-OVA, or control rabbit IgG, for example. All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

Quantitative assessments of staining intensity may be made using a morphometric photomicroscope. Translocation of alkaline protease into the serum can be detected by capture ELISA. To assess the importance of catalytic activity, AEBSF-inactivated alkaline protease can be given to mice by whatever protocol results in positive staining with the active protease.

The following routes of protease administration can be compared: Group 1 is injected by the intragastric route under conditions showing positive tissue staining. Group 2 receives protease in the drinking water. Protease can be dissolved in autoclaved tap water at a concentration of 100 μg/ml, and the amount of drinking water consumed daily can be recorded. It is estimated that a 25 g mouse usually consumes approximately 4 ml of water per day. Total volumes consumed can be recorded when the solutions are changed twice daily to minimize microbial contamination. Total mass of protease consumed may be calculated from these recordings. At that time aliquots of any remaining enzyme solutions may be frozen until the residual proteolytic activity can be determined. Group 3 receives a single i.p. 10 μg dose of the protease dissolved in PBS; and Group 4 is given a single dose of the protease (10 μg in PBS) by the i.v. route. Additional control groups can receive either distilled water or PBS, depending on the route of injection. Each group may consist of 8 mice, and the tissues listed above can be collected 1, 3, 6, and 9 hours post-challenge from 2 mice per time point. Once the optimum response times have been identified, a dose-response may be determined with four 3-fold increasing challenge doses that bracket these initial doses. Tissues are fixed, embedded, sectioned and stained with the appropriate anti-alkaline protease polyclonal antibody (one may use the antibodies from Example 9, for example). Staining intensity, distribution and quantization can be done as described herein. Serum concentrations of protease may be determined by capture ELISA.

Sufficient translocation of protease into the serum can follow bolus intragastric challenge allowing for an estimate of the total amount of transfer. Using mice challenged by the intraperitoneal route as controls, an estimate of the period of translocation can be made from the preceding studies. Then groups of mice can challenged i.p. or by gastric intubation with a single dose of protease found above to produce detectable serum levels of the protein. The animals are euthanized at six time points (e.g., 0.5, 1, 2, 4, 6, and 8 h). Blood is collected and sera separated and frozen until assay. The capture ELISA can be used to measure circulating levels of the protease and estimate total mass transferred based on a calculation of the area under the concentration-time curve.

Control sections of tissues may be fixed, stained with hematoxylin and eosin and examined for gross histopathological changes in the crypts, villous epithelium, and submucosa. Particular note should be made of the loss of cell-cell junctions, expression of apoptotic changes, epithelial sloughing, inflammatory cell infiltration, submucosal edema, and surface erosion/ulceration. Dose- and time-dependent changes can be noted and correlated with similar changes in Caco-2 monolayers.

### Example 14

### The Biochemical, Immunological and Catalytic Properties of Alkaline Proteases Distributed in Extragastrointestinal Tissues, Including the Blood Circulation

Proteases recovered from extra-intestinal sites (e.g., blood circulation) can be characterized in terms of their antigenic integrity, molecular mass, degree of proteolytic fragmentation, residual enzymatic activity and specificity, and association with plasma proteins (e.g., plasma protease inhibitors). This allows the person of ordinary skill in the art to distinguish between the transepithelial uptake and transfer of intact active proteases versus denatured enzymes or peptides derived from the digestion of these proteins.

Immunoprecipitation and Western blotting can be used to establish how much alkaline protease found in serum has undergone proteolysis and whether alkaline proteases are complexed with known plasma proteins (e.g., protease inhibitors). Immunoprecipitation followed by catalytic assays may be used to determine if the immunologically detectable proteins retain enzymatic activity. Co-localization studies with antibodies produced against the N and C termini of the proteases can ascertain if the proteins found in tissues are intact (e.g., full length). Catalytic histochemistry can be used to measure the activity and specificity of the enzymes associated with cell monolayers in vitro. All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

Two techniques are suitable to characterize blood-borne enzymes: (i) immunoprecipitation and Western blotting for determining molecular mass and association with serum proteins and (ii) modified protease capture assays for determining if these enzymes are catalytically active.

To ascertain associations with plasma protease inhibitors, proteases are immunoprecipiated and blotted onto membranes and the membranes probed with antibodies to the following plasma protease inhibitors: (i) α₂-macroglobulin (Novus); (ii) α₁antitrypsin (Biodesign); and (iii) C1 Inhibitor (Biodesign). The catalytic activity of any proteases immunoprecipitated from serum can then be determined by capturing the protease from serum and assaying the activity with a kinetic enzyme plate assay using chromogenic or fluorogenic peptide substrates.

A number of techniques and reagents have developed that enable detection of catalytically active proteases on or within living cells using synthetic fluorogenic peptide substrates (for instance, cathepsin B, D, H, K and L, dipeptidyl peptidase IV, and caspase 1, 3, 7, and 9) have been enzymatically detected within cells using peptides covalently linked to rhodamine 110, resofurin or cresyl violet. The specificities of Aspergillus oryzae alkaline protease and deuterolysin have been characterized (val-leu-lys and arg-val-arg-arg (SEQ ID NO: 10), respectively). Therefore, a number of Z-dipeptide-rhodamine 110 substrates available from commercial sources (Molecular Probes, Bachem, Novabiochem and Anaspec) that are related to these sequences can be tested, and the best cell-permeable dipeptide substrate (e.g., Z-pro-arg-rhodamine 110) can be selected. Data may be expressed as Δfluorescence units/min.

Then Caco-2 cell monolayers can be grown in 96-well microtiter plates and used to measure the hydrolysis of the appropriate rhodamine 110-labeled substrate after the cell monolayers are first allowed to take up and/or translocate the proteases. The specificity of the substrate hydrolysis may be determined by comparison of homologous and heterologous substrates, by the addition of the class-specific protease inhibitors (Sigma Protease Inhibitor Panel) or combining the labeled substrate with a 500 molar excess of homologous non-fluorescent dipeptides prior to its addition to the cells.

For determining the localization of fluorescent product and the co-localization of product with enzyme within the cells, Caco-2 cells are grown on coverslips. Monolayers can be exposed to the proteases under conditions that lead to their uptake or translocation and the rhodamine 110-labeled substrates can be added.

A similar approach can be taken with tissues from mice injected with proteases to establish para- or intracellular uptake of active proteases in the intestinal epithelium. A person of ordinary skill in the art can detect leukocyte esterases in paraformaldehyde-fixed tissue sections using the chromogenic substrate ASD-chloroacetate esterase.

Using the methods described herein and protocols known in the art, the person of ordinary skill in the art is able to immunoprecipitate circulating proteases with antibody-coated Sepharose beads. Evaluation of immunoprecipitated enzymes by Western blotting only requires 10-50 ng of protease, using high-titered, high-affinity antibodies. Combining N and C terminus-specific antibodies in immunoprecipitation-Western blotting experiments can enable a skilled artisan to determine if the enzymes are catalytically intact. Purified A. oryzae proteases are fairly stable for weeks at 4° C. and retain activity at −20° C. for several months. They do not undergo extensive autoproteolysis as evidenced by acquisition of additional bands on SDS-PAGE. Thus, additional bands not seen with the purified proteins prior to their injection can be taken as evidence of in vivo modifications. Both paracellular protease and intra-organelle proteases have been detected by this approach. All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 15

The Protective Effects of Alkaline Protease on the Induction of IBD in the Hapten-Induced Mouse Model when Alkaline Protease is Administered Systemically or by the Oral Route

This example uses the TNBS model in the SJL mouse, a susceptible strain, comparing systemic versus oral administration of the alkaline protease as a prophylactic treatment of blocking the induction of gastrointestinal inflammation. Endpoint measurements can include histopathological evaluation and scoring of the lesions as known in the art.

SJL strain mice (Jackson Labs) can be lightly anesthetized, and a solution of 25-50 μg of TNBS in 100 μl 50% ethanol can be administered by enema. Initial studies can determine whether sufficient pathology is induced by a single injection or whether the two challenge protocol (day 0 and day 7) is preferable. Response parameters include: (a) body weight measured on days 0, 2, 4, 6, 8, and 10; (b) pathological scoring of lesions in the intestine on day 10 (0=no inflam; 1=low level leukocyte infiltration; 2=medium infiltration, vascular intensity with some wall thickening; 3=high level infiltration, vascular response and thickening; 4=transmural infiltration, loss of goblet cells); (c) and immunohistological staining for activated caspase-3 and TNF-α. Wall thickening can be measured, for instance, as described by Dohi et al. (1999) “Hapten-induced colitis is associated with colonic patch hypertrophy and T helper cell 2-type responses.” J Expt Med 189: 1169-79, which is incorporated by reference. Mice can be maintained on normal mouse chow and water ad libitum throughout the study period. Sufficient numbers of mice per group should be studied to achieve the P<0.05 level of significance.

Mice can be treated with alkaline protease prior to or during the induction of colitis. Commencing with the TNBS challenge, mice can be injected i.p. (intraperitoneal) at 0, 12, 24, and 36 hours with 10 μg purified AP. Body weight can be monitored, and intestinal tissues (ileum, cecum and distal colon) can be taken at the end of the study period. Three groups, for example, can be compared: (a) Group 1 can be treated with protease but would not be challenged with TNBS; (b) Group 2 can be challenged with TNBS; (c) Group 3 can be challenged with TNBS and treated with AP. On day 7 (single injection of TNBS) or day 10 (TNBS given on day 0 and day 7), tissues can be recovered and fixed. Histopathological scoring can follow, for instance, that of Dohi et al. (1999) “Hapten-induced colitis is associated with colonic patch hypertrophy and T helper cell 2-type responses.” J Expt Med 189: 1169-79, which is incorporated by reference, and immunohistology for activated caspase-3 and TNF-α should follow standard procedures as known in the art.

Next, to assess oral dosage, Groups 1 and 2 can receive sufficient alkaline protease to achieve a total dose of 25-50 mg/kg in the drinking water over the entire 7-10 days of the TNBS challenge (determined by first measuring average daily water consumption). Enzyme-containing water can be replaced regularly to assure maintenance of enzymatic activity, and the protease activity monitored on a regular basis. Group 3 can receive four i.p. injections (10 μg each at 0, 24, and 48 h) of alkaline protease during this period. Group 4 should not receive protease. Groups 2-4 can be challenged with TNBS on the same day protease treatment is begun under conditions determined above for the optimal induction of colitis. Their tissues can be collected for the evaluation of histopathological scoring, and TNF-α and caspase expression. Body weights can be monitored.

Both the i.p. and oral alkaline protease treatments can prevent and/or lessen weight loss and IBD induction by TNBS. An oral alkaline protease dose of 25-50 mg/kg may be sufficient to prevent IBD even when provided over a 7-10 d period. The person of ordinary skill in the art can test higher doses up to 100 mg/kg (the equivalent of 1000 mg per day for 7 days for a 70 kg human). All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 16

### Alkaline Protease can Lessen Intestinal Lesions when Administered to Mice with Established Inflammatory Bowel Disease (IBD)

Alkaline protease can reverse and/or lessen established IBD using the hapten model in SJL mice.

SJL mice can be given TNBS by enema for the induction of colitis, and their body weights can be monitored every 3 days. When there has been a decrease in body mass by at least 15% (typically day 7), the animals can be given either four i.p. injections of 10 μg alkaline protease every 12 hours (Group 1) or administered alkaline protease in their drinking water at a concentration found above to decrease IBD parameters (Group 2). A control group can be challenged with TNBS, but would not receive protease (Group 3). Body weights can then be monitored daily, and at the end of one week, mice are euthanized and tissues taken for scoring the various histopathological and immunohistochemical parameters as known in the art.

### Example 17

### Alkaline Protease can Treat IBD in Mice that Spontaneously Develop the Disease

C3H/HeJBir mouse (Jackson Labs) can be used to show that alkaline protease therapy can correct spontaneous colitis in the mouse. This particular mouse strain develops a transmural Th1-, cytokine-dependent colitis similar to CD that begins at 3-6 wk of life and gradually wanes until it resolves at 3 months of age. T cells and Th1 cytokines (IL-112, IFN-γ and TNF-α) mediate the disease which is maintained by an ongoing reaction to microbial flora antigens.

C3H/HeJBir mice can be obtained at 5-6 wk of age from Jackson Labs. Mice can be divided into three groups, for example: Group 1 should receive no further treatment and may simply be monitored for clinical signs, including daily measurements of body weight and endpoint pathology. Group 2 should be administered alkaline protease in their drinking water at a dose of 10-25 mg/kg/week for one week. Group 3 should be administered alkaline protease in their drinking water (dose determined above) for two weeks. After 1 week (Group 2 and half of Group 1) or 2 weeks (Group 3 and half of Group 1), animals can be sacrificed and body weight changes and histopathological scores of inflammation and tissue damage (leukocyte infiltration, ulceration, abscesses and fibrosis), activated caspase-3 expression and TNF-α expression can be measured by methods known in the art.

Alkaline protease can correct the weight loss and the pathological lesions seen in these mice during weeks 6-10 of life. IBD in this model is concentrated in the cecum and ascending colon (right sided). All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 18

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 19

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment according to the lessening of the symptoms and/or the development of side effects.

### Example 20

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 21

### Treatment of Ulcerative Colitis

A 70 kg human patient diagnosed with ulcerative colitis can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 22

### Treatment of Ulcerative Colitis

A human patient diagnosed with ulcerative colitis can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 23

### Treatment of Ulcerative Colitis

A 70 kg human patient diagnosed with ulcerative colitis can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 24

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 25

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 26

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 27

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 28

### Prophylaxis of Ulcerative Colitis

A human patient diagnosed with risk factors for ulcerative colitis can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 29

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 30

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 31

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 32

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 33

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 34

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 35

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 36

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 37

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 38

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 39

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 40

### Treatment of Inflammatory Bowel Disease (IBD)

A human patient diagnosed with inflammatory bowel disease (IBD) can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 41

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 42

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 43

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 44

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 45

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 46

### Prophylaxis of Inflammatory Bowel Disease (IBD)

A human patient diagnosed with risk factors for inflammatory bowel disease (IBD) can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 47

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 48

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 49

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 50

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 51

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 52

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 53

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 54

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 55

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 56

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 57

### Treatment of Crohn's Disease

A human patient diagnosed with Crohn's disease can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 58

### Treatment of Crohn's Disease

A human patient diagnosed with Crohn's disease can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 59

### Treatment of Crohn's Disease

A human patient diagnosed with Crohn's disease can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 60

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 61

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 62

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 63

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 64

### Prophylaxis of Crohn's Disease

A human patient diagnosed with risk factors for Crohn's disease can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 65

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 66

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 67

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 68

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 69

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 70

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 71

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 72

### In Vitro Assay for Increased Uptake of Agent Co-Administered with Alkaline Protease

The uptake of an agent with and without alkaline protease can be tested by using an in vitro uptake model. Caco-2 cells are prepared as taught in Example 12 and the agent is added in the presence and absence of alkaline protease. After Caco-2 monolayers have reached confluence in dual chamber transwell assay plates, the culture medium can be replaced with KRB, and initial TER may be measured after a one hour stabilization period. Then different donor chambers receive test compounds and controls including three-fold dilutions of each of the A. oryzae proteases in the range of 0.1-10 μM concentrations (3-300 μg/ml for alkaline protease) and Test compound (agent). HRP may serve here as a nonprotease control enzyme that is normally excluded by the monolayer. At 20 minute intervals up to 180 min, 501 samples can be taken from the donor (apical) and acceptor (basolateral) compartments and frozen at −80° C. until assay. Fresh KRB is added back to the chambers, and calculations of the concentrations of test proteins can be adjusted for this add-back dilution. At the end of the period of protease exposure, test samples can be removed and replaced with KRB, and cell monolayer TER is measured for each well 10 and 60 minutes later. Agent and protease translocation from donor to acceptor compartments may be determined by known enzymatic and immunological assays. The levels of the agent which cross the Caco-2 monolayer are compared in the presence and absence of alkaline protease. Increased levels of agent in the presence of alkaline protease is indicative of an increased level of uptake due to co-administration with alkaline protease.

### Example 73

### In Vivo Assay for Increased Uptake of Agent Co-Administered with Alkaline Protease

Mice can administered a composition comprising alkaline protease and agent by being injected i.p. (intraperitoneal) at 0, 12, 24, and 36 hours with a composition comprising 10 μg of purified alkaline protease. Four groups, for example, can be compared: (a) Group 1 can be administered a placebo (a composition with vehicle only) via injection; (b) Group 2 can be administered via injection a composition comprising 10 μg alkaline protease; (c) Group 3 can be orally administered a composition comprising the test agent; and (d) Group 4 can be administered via injection a composition of 10 μg alkaline protease and, orally, the test agent. Alternatively, the mice can be injected once a day for 10 days.

Alternatively, to assess oral dosage, Groups 2 and 4 can receive sufficient alkaline protease to achieve a total dose of 25-50 mg/kg in the drinking water over the entire 7-10 days (determined by first measuring average daily water consumption). Enzyme-containing water can be replaced regularly to assure maintenance of enzymatic activity, and the protease activity monitored on a regular basis. Groups 1 and 3 can receive their compositions orally as well using a similar methodology and described herein. Body weights can be monitored.

On each day for 7-10 days, tissues can be recovered and fixed by using methods taught herein and those known in the art. Additionally, blood samples may be taken every day to assess the amount of alkaline protease and/or agent which has been taken into the blood stream and measured using methods as taught herein or known in the art (e.g., Western Blot and ELISA). The blood and tissue samples are compared and those showing an increased level of the test agent in the blood and tissues where it was co-administered with alkaline protease (either injected or orally) is indicative of increased transport.

### Example 74

### Enhanced Uptake of Adjuvant via Co-Administration with Alkaline Protease

A human patient receiving a vaccine in conjunction with an adjuvant can be administered a pharmaceutical composition comprising alkaline protease and an effective dose of an immunogen accompanied by an adjuvant. The human patient is orally administered a pharmaceutical composition comprising alkaline protease and an effective dose of an immunogen accompanied by an adjuvant, wherein it is formulated to provide 25-50 mg/kg of alkaline protease. The human patient is monitored for an immune response and treated according to the development of side effects. In addition, the human patient can be tested for the presence of alkaline protease and the adjuvant in the blood to measure the enhanced uptake.

### Example 75

### Enhanced Uptake of Adjuvant via Co-Administration with Alkaline Protease

A human patient receiving a vaccine in conjunction with an adjuvant can be administered a pharmaceutical composition comprising alkaline protease and an effective dose of an immunogen accompanied by an adjuvant. The human patient is orally administered a pharmaceutical composition comprising alkaline protease and an effective dose of an immunogen accompanied by an adjuvant, wherein it is formulated to provide 25-50 mg/kg of alkaline protease, and wherein said protease provides activity which cleaves Val-leu-lys-pNA at least about 100 mOD/min. The human patient is monitored for an immune response and treated according to the development of side effects. In addition, the human patient can be tested for the presence of alkaline protease and the adjuvant in the blood to measure the enhanced uptake.

### Example 76

### Enhanced Uptake of Antibiotic Via Co-Administration with Alkaline Protease

A human patient receiving an antibiotic can be administered a pharmaceutical composition comprising alkaline protease and an effective dose of antibiotic. The human patient is orally administered pharmaceutical composition comprising alkaline protease and an effective dose of antibiotic, wherein the composition is formulated to provide 25-50 mg/kg of alkaline protease. The human patient is monitored for the presence of alkaline protease and the antibiotic in the blood to measure the enhanced uptake and/or treated for the development of side effects.

### Example 77

### Enhanced Uptake of Antibiotic Via Co-Administration with Alkaline Protease

A human patient receiving an antibiotic can be administered a pharmaceutical composition comprising alkaline protease and an effective dose of antibiotic. The human patient is orally administered a pharmaceutical composition comprising alkaline protease and an effective dose of antibiotic, wherein said composition is formulated to provide 25-50 mg/kg of alkaline protease, and wherein said protease provides activity which cleaves Val-leu-lys-pNA at least about 100 mOD/min. The human patient is monitored for the presence of alkaline protease and the antibiotic in the blood to measure the enhanced uptake and/or treated for the development of side effects.

### Example 78

### Enhanced Uptake of Antibody Via Co-Administration with Alkaline Protease

A human patient receiving an antibody can be administered a pharmaceutical composition comprising alkaline protease and an effective dose of antibody. The human patient is orally administered pharmaceutical composition comprising alkaline protease and an effective dose of antibody, wherein the composition is formulated to provide 25-50 mg/kg of alkaline protease. The human patient is monitored for the presence of alkaline protease and the antibody in the blood to measure the enhanced uptake and/or treated for the development of side effects.

### Example 79

### Enhanced Uptake of Antibody Via Co-Administration with Alkaline Protease

A human patient receiving an antibody can be administered a pharmaceutical composition comprising alkaline protease and an effective dose of protein. The human patient is orally administered a pharmaceutical composition comprising alkaline protease and an effective dose of antibody, wherein said composition is formulated to provide 25-50 mg/kg of alkaline protease, and wherein said protease provides activity which cleaves Val-leu-lys-pNA at least about 100 mOD/min. The human patient is monitored for the presence of alkaline protease and the antibody in the blood to measure the enhanced uptake and/or treated for the development of side effects.

### Example 80

### Enhanced Uptake of Peptide Via Co-Administration with Alkaline Protease

A human patient receiving a peptide can be administered a pharmaceutical composition comprising alkaline protease and an effective dose of peptide. The human patient is orally administered pharmaceutical composition comprising alkaline protease and an effective dose of peptide, wherein the composition is formulated to provide 25-50 mg/kg of alkaline protease. The human patient is monitored for the presence of alkaline protease and the peptide in the blood to measure the enhanced uptake and/or treated for the development of side effects.

### Example 81

### Enhanced Uptake of Peptide Via Co-Administration with Alkaline Protease

A human patient receiving a peptide can be administered a pharmaceutical composition comprising alkaline protease and an effective dose of protein. The human patient is orally administered a pharmaceutical composition comprising alkaline protease and an effective dose of peptide, wherein said composition is formulated to provide 25-50 mg/kg of alkaline protease, and wherein said protease provides activity which cleaves Val-leu-lys-pNA at least about 100 mOD/min. The human patient is monitored for the presence of alkaline protease and the peptide in the blood to measure the enhanced uptake and/or treated for the development of side effects.

### Example 82

### Enhanced Uptake of a Protein Via Co-Administration with Alkaline Protease

A human patient receiving a protein can be administered a pharmaceutical composition comprising alkaline protease and an effective dose of protein. The human patient is orally administered pharmaceutical composition comprising alkaline protease and an effective dose of protein, wherein the composition is formulated to provide 25-50 mg/kg of alkaline protease. The human patient is monitored for the presence of alkaline protease and the protein in the blood to measure the enhanced uptake and/or treated for the development of side effects.

### Example 83

### Enhanced Uptake of Protein Via Co-Administration with Alkaline Protease

A human patient receiving a protein can be administered a pharmaceutical composition comprising alkaline protease and an effective dose of protein. The human patient is orally administered a pharmaceutical composition comprising alkaline protease and an effective dose of protein, wherein said composition is formulated to provide 25-50 mg/kg of alkaline protease, and wherein said protease provides activity which cleaves Val-leu-lys-pNA at least about 100 mOD/min. The human patient is monitored for the presence of alkaline protease and the protein in the blood to measure the enhanced uptake and/or treated for the development of side effects.

Although the invention has been described in some detail by way of illustration and example for purposes of clarity of understanding, it will be obvious that certain changes and modifications may be practiced within the scope of the appended claims. Modifications of the above-described modes for carrying out the invention that are obvious to persons of skill in medicine, pharmacology, microbiology, and/or related fields are intended to be within the scope of the following claims.

All publications and patent applications mentioned in this specification are indicative of the level of skill of those skilled in the art to which this invention pertains. All such publications and patent applications are herein incorporated by reference to the same extent as if each individual publication or patent application was specifically and individually indicated to be incorporated by reference.

1. A method of facilitating transport of an agent across a mucosal membrane in a patient comprising administering a composition comprising an alkaline protease and an agent to a patient, wherein the uptake of the agent is increased relative to uptake in the absence of said alkaline protease.

2. The method of claim 1, wherein said agent is an adjuvant, antibiotic, antibody, antigen, diagnostic agent, DNA vaccine, drug, gene-delivery vector, gene, macromolecule, nanoparticle, nucleic acid, ribonucleic acid, peptide, peptide vaccine, prodrug, protein, or vaccine.

3. The method of claim 1, wherein said alkaline protease increases the bioavailability of the agent.

4. The method of claim 1, wherein said composition is administered orally.

5. The method of claim 1, wherein said isolated alkaline protease is an Aspergillus oryzae alkaline protease.

6. The method of claim 1, wherein said composition does not include a 26 kDa protease or deuterolysin.

7. The method of claim 5, wherein said isolated Aspergillus oryzae alkaline protease comprises SEQ ID NO: 2.

8. The method of claim 5, wherein said isolated Aspergillus oryzae alkaline protease is recombinantly produced.

9. The method of claim 1, wherein said composition comprises a pharmaceutically acceptable carrier, excipient, diluent, or solution.

10. The method of claim 1, wherein said composition is a food supplement, a nutritional supplement, or a food product.

11. Use of a composition comprising an alkaline protease and an agent to facilitate transport of the agent across a mucosal membrane comprising administering wherein the uptake of the agent is increased relative to uptake in the absence of the alkaline protease.

12. An isolated antibody or antigen-binding fragment which specifically binds to alkaline protease.

13. The antigen-binding fragment of claim 12, wherein said antigen-binding fragment is F(ab)₂, Fab, Fv, and sFv.

14. The antibody of claim 12, wherein said antibody is IgG, IgD, IgE, IgA, or IgM.

15. The antibody or antigen-binding fragment of claim 12, wherein said alkaline protease is Aspergillus oryzae.

16. The antibody or antigen-binding fragment of claim 12, wherein said antibody or antigen-binding fragment is chimeric, single-chain, or humanized.

17. The antibody or antigen-binding fragment of claim 12, wherein said antibody is monoclonal or polyclonal.

18. The antibody or antigen-binding fragment of claim 12, wherein said antibody or antigen-binding fragment binds to SEQ ID NO: 2.

19. A composition comprising the antibody of claim 12.

20. The composition of claim 19, wherein said composition is a pharmaceutical composition.
